(19) United States
(12) Patent Application Publication
(10) Pub. No.: US 2026/0002112 A1
(43) Pub. Date: Jan. 1, 2026

(54) SYSTEM FOR NON-PERTURBATIVE SAMPLING OF SAMPLE-VOLUME-LIMITED BIOREACTORS

(71) Applicant: VANDERBILT UNIVERSITY, Nashville, TN (US)

(72) Inventors: John P. Wikswo, Brentwood, TX (US); Ronald S. Reiserer, Nashville, TN (US); Kyle Hawkins, Nashville, TN (US)

(21) Appl. No.: 19/323,144

(22) Filed: Sep. 9, 2025

Related U.S. Application Data
(63) Continuation-in-part of application No. 18/527,801, filed on Dec. 4, 2023, now abandoned.
(60) Provisional application No. 63/429,691, filed on Dec. 2, 2022.

(51) Int. Cl. C12M 1/34; C12M 1/00; C12M 1/06

(52) U.S. Cl. CPC C12M 41/44; C12M 27/02; C12M 29/14; C12M 41/26; C12M 41/32; C12M 41/34

(57) ABSTRACT

A non-perturbative sampling system includes a bioreactor comprising a chamber containing media with cells; an input tube for delivering nutrient-laden media to the chamber; and an output tube for withdrawing a sample from the chamber; an input pump configured to deliver the nutrient-laden media at an inflow rate; an output pump configured to withdraw the sample at an outflow rate; and a controller configured to operate the input and output pumps to regulate the inflow and outflow rates, respectively, such that the bioreactor is a variable-volume bioreactor in which an instantaneous volume of media either varies continuously with time or is held constant, and operable in sample accumulation phase, sample storage phase, sample withdrawal phase, or volume restoration phase, to maintain the quantity of nutrients per cell within the media in the chamber unchanged. The sample withdrawal phase either follows the sample accumulation phase or precedes the volume restoration phase.

### CROSS-REFERENCE TO RELATED PATENT APPLICATIONS

This application is a continuation in part application of U.S. patent application Ser. No. 18/527,801, filed Dec. 4, 2023, which itself claims priority to and the benefit of U.S. Provisional Patent Application Ser. No. 63/429,691, filed Dec. 2, 2022, which are incorporated herein by reference in their entireties.

### STATEMENT AS TO RIGHTS UNDER FEDERALLY-SPONSORED RESEARCH

This invention was made with government support under Grant No. 2117782 awarded by the National Science Foundation. The government has certain rights in the invention.

### FIELD OF THE INVENTION

The invention relates generally to bioreactors, and more particularly to a system for non-perturbative sampling of small bioreactors and applications thereof.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The Need for Arrays of Small Bioreactors that Support Non-Perturbative Sample Accumulation, Storage, and Removal

A bioreactor is any vessel in which living cells are cultured in a constrained volume. There is an ever-growing interest in the use of bioreactors to study the biological production of target molecules that are of medical, technological, environmental, or scientific value, such as sugars, alcohols, proteins, antibodies, short-chain fatty acids, and other biochemicals that can be produced by appropriately engineered Saccharomyces cervisiae, Chinese hamster ovary (CHO) cells, or other mammalian, microbial, fungal, or algal cells. Given the need to characterize quickly a large number of different cells, the trend has been to use arrays of smaller and smaller bioreactors configured as well plates. Early well plates might have had 12 wells with working volumes of 2 mL and a 96-well plate could have a working volume of 150 μL. Today, a 1,536-well plates have working volumes of 8 μL. While well plates with their small volumes may be the endpoint for many biological assays, particularly in toxicological studies, they are also used as models for larger production bioreactors, which have volumes of 1 to 10,000 L. Intermediate between these small and large bioreactors are a number of mesoscale systems that culture cells in volumes between 2 and 500 mL using a wide variety of formats and stirring/mixing schemes, including shaken flasks and bags. In another application, it is useful to use a perfused well plate or other miniature bioreactor system operating in batch of fed-batch mode to study the steady-state metabolic activity of cells, for example to determine the effects of genetic modifications of a strain of yeast on its metabolic activity. In these cases, it is important to be able to remove sequentially a series of small samples of media or cells for analysis or add media to replace the removed media and its metabolized nutrients.

As an example, consider the culture of cells in a 96-well plate where each well “bioreactor” has a working volume of 150 μL. If 50 μL is the minimum volume of media required to analyze the metabolic activity of the cells in that well, then the removal of 50 μL for analysis and its replacement with 50 μL of fresh media could result in a 33% perturbation of the concentrations of the cellular metabolites in the well, representing a significant change in the biochemical and/or mechanical microenvironment to which the cells are exposed. The addition of a single droplet of nutrient media with a volume of 20 μL could represent a 13.3% change in the concentration of nutrients or metabolites. These changes would be considered significant, since they may lead to irreversible changes in gene expression in the cultured cells.

As another example, were the bioreactor to have a volume of 15 mL, then the perturbations of a 50 μL fluid exchange or the addition of a 20 μL nutrient droplet would represent only 0.3% and 0.1% effect, respectively, which in most cases might be metabolically insignificant. For a 250 mL bioreactor, the changes would be 0.02% and 0.008%, respectively, and would generally be considered negligible. However, were the bioreactor to have a volume of only 1.5 mL, the changes from 50 μL and 20 μL would be 3.3% and 1.3%, which could be metabolically significant. Obviously the significance of the volume change depends upon three factors, the volume of the bioreactor, the volume of the sample, and the acceptable effect threshold expressed as a percentage of the bioreactor volume. For the purposes of this invention, we define a sample-volume-limited (SVL) bioreactor as any bioreactor for which the intended sample volume represents a larger than acceptable percentage of the bioreactor volume. Should the percentage be unacceptable, the user could either devise an assay that required a smaller sample volume, use a larger bioreactor, or, accumulate more sample. Should serial samples be required, the minimum time to acquire adequate sample volume will also be determined by the perfusion or refresh rate of any SVL bioreactor. Given the current state of bioreactor analytics, a sample volume of 0.5 mL would be typical for untargeted transcriptomic characterization and 2 mL would be put to good use. As shown in Table 1, with the high effect threshold of 10% and a sample volume of 100 μL, any bioreactor whose volume is less than 2 mL would definitely be an SVL bioreactor.

TABLE 1
Bioreactor volume consistent with a stated sample volume (rows)
and acceptable sampling effect from sample removal (columns).

| | | | Percent Acceptable Effect from Sample Removal | | | | | | | | | | | | | | |
|---|---|---|---|---|---|---|---|---|---|---|---|---|---|---|---|---|---|
| | | | 0.1% | | 0.2% | | 0.5% | | 1% | | 2% | | 5% | | 10% | | |
| Sample | 10 | μL | 10 | mL | 5 | mL | 2 | mL | 1 | mL | 500 | μL | 200 | μL | 100 | μL | 10 μL Removal |
| Volume | 20 | μL | 20 | mL | 10 | mL | 4 | mL | 2 | mL | 1 | mL | 400 | μL | 200 | μL | 20 μL Droplet |
| | 50 | μL | 50 | mL | 20 | mL | 10 | mL | 5 | mL | 2.5 | mL | 1 | mL | 500 | μL | |
| | 100 | μL | 100 | mL | 50 | mL | 20 | mL | 10 | mL | 5 | mL | 2 | mL | 1 | mL | Untargeted MS |
| | 200 | μL | 200 | mL | 100 | mL | 40 | mL | 20 | mL | 10 | mL | 4 | mL | 2 | mL | |
| | 500 | μL | 500 | mL | 250 | mL | 100 | mL | 50 | mL | 25 | mL | 10 | mL | 5 | mL | Transcriptomics |
| | 1 | mL | 1 | L | 500 | mL | 200 | mL | 100 | mL | 50 | mL | 20 | mL | 10 | mL | |
| | 2 | mL | 2 | L | 1 | L | 400 | mL | 200 | mL | 100 | mL | 40 | mL | 20 | mL | |
| | 5 | mL | 5 | L | 2 | L | 1 | L | 500 | mL | 250 | mL | 100 | mL | 50 | mL | |
| | 10 | mL | 10 | L | 5 | L | 2 | L | 1 | L | 500 | mL | 200 | mL | 100 | mL | Four × 2 mL |

per day

It is important to recognize that well plates with working volumes of 1 to 2 mL will be considered SVL bioreactors unless the sample volume is less than 10 μL and volume-associated changes greater than 2% are acceptable. This invention allows the accumulation of larger sample volumes without metabolic effects associated with sampling-related volume changes. with

Considering a particular application of small bioreactors, we recognize that the development of an optimized cell line to produce the desired chemical presents significant challenges. The sequence of steps in this process is as follows: typically, well-established procedures are used to genetically engineer already proven production cell line by attempting to add, remove, or modify the gene(s) necessary for producing the desired biomolecule. The desired changes are made to a population of cells, with significant variations in the effectiveness of the changes between individual cells due to uncertainties in the transfection process. In particular, the metabolic behavior of a particular engineered clone may depend upon whether that cell is grown at low cell density in a well plate, which can be readily done using high-throughput screening (HTS) technologies, or at high cell density in a production-scale bioreactor, which is time-consuming and extremely expensive.

Using conventional high-throughput well plate technologies, a thousand clones can be produced for approximately $1 each, but few of these will produce the requisite quantity of the chemical product when the cells are cultured in large thousand-liter production bioreactors, whose levels of nutrients, waste products, growth factors, dissolved oxygen and carbon dioxide, temperature and pH will be different from that in the well plate and even vary between multiple zones within the large bioreactor. In the large bioreactors, cells will experience different residence times in the various zones, producing temporal variations in the microenvironments to which the cells are exposed that will result in changes in cellular gene expression profiles that can last far longer than the time required for a cell to transit any single zone.

Existing assays using arrays of a thousand microfluidically controlled nanopens or 96, 384, or more wells in a well plate are limited their ability to predict which clone will scale best. In some applications, single cells selected from a particular nanopen are expanded into a clonal population using an array of 24 or 48 15 mL bioreactors. One of the best platforms for predicting how a particular cell line will perform at production scale is the 250 milliliter (mL) reactor, but a single 250 mL run can cost approximately $1,000 for cells, reagents, and personnel, severely limiting the number of clonal populations that can be evaluated fully. Under current practice, the initial screening for scale-up may be limited to as few as 12 clonal populations. If the selected clones fail to scale, the 250 mL process must be repeated, thereby increasing development costs and delaying the time-to-market for the target product. As result, iterations to engineer optimized clones require significant time and money.

One challenge addressed by this invention is that results obtained at low cell densities in a nanopen or well plate may not be predictive of cellular performance in a high-cell-density production bioreactor. Hence, once the original population of cells has been transfected, it is imperative to select from thousands of different parent cells a smaller number of clones that might sustain their appropriate metabolic activity and product production at ever-increasing bioreactor volumes. The probability of correctly identifying from that subset a clone optimized for production-scale culture can be increased by culturing a large number of clones in small bioreactors that can replicate the range of conditions encountered by cells in production bioreactors.

Proper metabolomic and transcriptomic characterization of each of these subset populations of suspended cells may require 0.5 mL or more of cell-containing media. As stated previously, removal of a 0.5 mL sample from a 15 mL bioreactor represents a 3% perturbation in bioreactor volume, where a removal of that volume from a 250 mL bioreactor is only a 0.2% perturbation. However, the removal of a one or more 0.5 mL samples from a 1.5 mL bioreactor represents a 33% reduction in volume with each withdrawal, which can disrupt the performance of the remaining cells and reduce the accuracy with which the small bioreactor adequately recapitulates the conditions found in the much larger production bioreactor. The size of the sample will also limit how quickly a second sample can be withdrawn to characterize how the cellular metabolism and gene expression are varying over time. The required size of a sample may be determined by the density of cells in the bioreactor: at low cellular concentrations, larger samples could be required than at higher cellular concentrations. For these reasons, the number of serial samples required during metabolic characterization and cell- and media-optimization is determined by the level of metabolic activity of the cells and the rate of change and stability of this activity.

Because the cells under test are replicating and producing their chemical product at a limited rate, it will take time to accumulate from a small bioreactor a sufficiently large sample to quantify the performance of that particular cell line. The obvious approach would be to collect serial aliquots of cells and media into a separate well, vial, or reservoir for future delivery to the appropriate analytical instrument, but were the cells and/or their metabolites in the sample to remain metabolically active while being collected, their metabolic activity, gene expression, and product-production profiles would ultimately reflect not the environment in the original small bioreactor but that of the smaller sample-collection container into which they are gathered. The aliquots could be frozen immediately upon delivery to the collection well, vial, or reservoir, but then it would be necessary to thaw these cells and the media that contains them prior to analysis, which would both take time and result in resumption of cellular metabolic activity at a rate determined by the uniformity and rate of thawing. An alternative approach used in tissue pathology would be to transiently heat the incoming sample to cross-link the proteins, but that would kill the cells and chemically modify the metabolites already present in the sample. The current invention addresses this problem by accumulating the sample internal to the SVL bioreactor with active control of the bioreactor parameters to ensure that the microenvironment encountered by all cells in the small bioreactor remains constant over the interval required to accumulate the requisite sample volume. After the sample is removed from the bioreactor, the bioreactor parameters are proportionally adjusted to ensure that the remaining cells continue to be cultured without perturbation of their microenvironment.

For this process to be efficient and address the problem of identifying and engineering an optimal clone, it is useful for this process to be conducted on hundreds or even a thousand separate SVL bioreactors, each of which has appropriate sensors and controls to adjust multiple bioreactor parameters. The controller that can accomplish this can also operate over a range of volumes, thereby supporting accurate control of bioreactor parameters during expansion of the selected clone from well plate to production volumes.

In summary, increasing the efficiency with which cells in production bioreactors convert input nutrients to output biological products is extremely important for minimizing the use of valuable input feed stocks while maximizing the amount of product produced. This leads to a pressing need for instruments that can operate a thousand independent or coupled 1 to 2 mL bioreactors in a manner that allows non-perturbative removal of samples and supports accurate scaling predictions with fewer cells and hence less time to expand a single cell into a clonal population. Such an instrument would require smaller volumes of growth factors, specialized nutrients, and other expensive chemicals, smaller physical space, and less effort by technical staff. The savings in time, space, and personnel by replacing a small number of 15 or 250 mL bioreactors with a larger number of 1 to 2 mL bioreactors that supports detailed parallel evaluation of large numbers of different clones will be significant. This invention addresses the non-perturbative accumulation, storage, and transfer of analytical samples from one or more SVL bioreactors by means of real-time sensing and control of bioreactor fluid input composition and flow, the rates of fluid output, media mixing, and gas exchange, and the adjustment of other bioreactor operational parameters. One application of this invention would be the rapid screening of a plurality of cell lines to identify the particular cell line that will be most productive in a large bioreactor whose operation is being replicated by the smaller, test bioreactors. The ability to remove samples from the SVL bioreactor without perturbing their biochemical, mechanical, and otherwise operational microenvironments would ensure that the SVL bioreactors accurately recapitulated the larger ones despite sample removal. Similarly, the ability to accumulate a sample over a lengthy period of time without metabolic alterations to the sample during accumulation is of great importance.

Types of Bioreactors for which this Invention is Applicable

In practice, SVL bioreactors can be used either for their own purposes or as models of several classes of larger bioreactors, including batch bioreactors, fed-batch bioreactors, continuous-perfusion bioreactors, and chemostats. In a batch bioreactor, the reactor is filled with nutrient-containing media and a small number of cells is introduced that consume nutrients, increase in number, and secrete the desired biological products until the nutrients are exhausted and/or the level of secreted metabolic inhibitors drive the cells to senescence or death. At the end of the batch, the reactor is emptied and the cells and media of the reactor are harvested to obtain the desired product chemical.

If the cells are adherent to the interior surfaces of the bioreactor or otherwise captured within the reactor, it is possible to replace media at a regular rate without losing the cells, i.e., by creating an intermittent (fed-batch) or continuous-perfusion bioreactor. In fed-batch mode, the bioreactor is initially filled to a fraction of its total volume with fresh media and then seeded with cells. As the cell population expands, the cells consume nutrients and their growth rate diminishes. Fresh media is then added to increase the volume of media in the reactor, and the existing cells receive additional nutrients. Again, as the cell population expands and more product is produced, the cells consume nutrients, and the growth and product-production rates diminish. This process of intermittent or continuous feeding, often with a varying feed rate, continues until the bioreactor is completely filled. At the end of the process, after the nutrients that were last added are consumed and product production slows, the cells and their products are harvested.

In continuous-flow bioreactors, the volume of the reactor can be held constant, and as media is added, media is also removed, either continuously or intermittently. Unless the cells are adherent to the bioreactor, captured, or otherwise retained, the addition of fresh media and the removal of spent media will lead to the loss of cells, possibly at a significant economic cost. The associated removal of suspended cells is either minimized by using tangential flow filtering or allowing transient cell settling immediately prior to media removal, or by using alternating tangential flow filtration or a comparable means to return the cells to the bioreactor, thereby maintaining a high concentration of metabolically active cells in the bioreactor to improve the economic return of the entire process. If adherent cells are grown on suspended carrier beads, these beads will either have to be restrained from leaving the bioreactor or collected upon exit and returned to the bioreactor.

Chemostats represent a subset of continuous-flow bioreactors used heavily to grow and study prokaryotic microbes, yeast, or suspended mammalian or other eukaryotic cells. In a chemostat, media is added continuously at a constant rate, and the removal of media and cells at the same rate ensures that the bioreactor maintains a constant volume. As long as the media replacement rate is below a particular threshold determined by the metabolic properties of the cell, the cell density and the surrounding chemical microenvironment will remain constant, hence the name “chemostat.” In operation, chemostats provide a steady supply of both fresh, cell-free, nutrient-laden media and oxygen (for cells with aerobic metabolism) that support cell growth and division within the bioreactor. An output tube is configured to limit the volume of media within the reactor, and some means for stirring/mixing and oxygenation ensures that the media within the bioreactor is well mixed and uniformly oxygenated. Once an empty bioreactor is filled to its maximum allowed volume, the constant-volume requirement implies that the steady-state rates of liquid inflow and outflow must be identical.

Because the chemostat is well mixed, the liquid outflow removes from the reactor not only conditioned media but also cells. The rate at which cells are removed from the reactor is determined by both the outflow rate and the concentration of cells in that outflow. The rate at which the number of cells within the reactor increases is determined by the number of cells in the bioreactor and their rate of division, which in turn is determined by their genetics and metabolic state and the concentration of nutrients and oxygen in the reactor. As the number of cells in the reactor increases, so does their rate of consumption of nutrients and oxygen and their production of metabolites and carbon dioxide. The higher the concentration of nutrients and oxygen in the reactor, the faster the cells will be able to grow and divide, up to some biological limit; similarly, the lower the concentration of nutrients and oxygen and the higher the concentration of growth-inhibiting metabolic waste products, the slower the cells grow, where below a critical value, they either enter into senescence or die.

Upon initial seeding of a chemostat, the number of cells in the reactor will increase until the rate of cell addition through cellular division equals the rate of their removal in the effluent, at which point the system reaches a chemical steady state, i.e., the bioreactor begins to function as a chemostat. Given that the cells require time, nutrients, and oxygen to grow and divide, for any particular input nutrient concentration and level of oxygen in the bioreactor there is a maximum rate at which the cells can reproduce, and hence there is a maximum input flow rate, above which all cells are eventually washed out of the reactor. If a higher concentration of cells is desired, the increased consumption of nutrients and oxygen and production of metabolic products from the growing number of cells can be offset by increasing the concentration of nutrients in the media, the rate of oxygen delivery, and the media replacement rate, up to the biological limit imposed by the minimum time a cell requires to grow and divide as compared to the residence time of the media in the reactor. For too rapid media replacement, cells will be washed out before they have the time to divide. This entire process can be described quantitatively by what is known as the Monod equation. One of the challenges in the field is to recapitulate in a SVL bioreactor the conditions encountered in larger bioreactors, with the realization that removal of samples and replacement of media may have vastly greater consequences in an SVL bioreactor than a large one, and that sample accumulation will be slower for an SVL bioreactor than a large one.

Analysis of Samples Withdrawn from a Bioreactor

The monitoring of the state of the cells in a bioreactor can be readily accomplished by measurement of the pH, optical density (OD), the concentrations of dissolved oxygen and carbon dioxide, and changes in the concentration of glucose, lactate, ammonia, and/or other metabolites in the bioreactor effluent as compared to the input media. All of these measurements can be accomplished either non-invasively within the bioreactor or from the effluent stream or a withdrawn sample. The optimization of the biomolecular production efficiency of a cell line growing in a bioreactor typically requires additional, quantitative, off-line analysis of the cellular secretome, i.e., the metabolites and other biomolecules secreted by the cells during culture, as well as the cellular proteome, metabolome, lipidome, and transcriptome, which in turn can be used to guide genetic modifications of the cells to enhance biomolecular production rates or expand the range of bioreactor conditions favorable for culturing a particular cellular phenotype. The probability of identifying a cell with appropriate gene expression and determining the nutrient concentrations that are optimal for a particular production objective can be increased by examining a large number of individual clonal cell populations, which motivates the drive for creating smaller and smaller bioreactors that can be used in larger and larger numbers, e.g., by using multi-well microchemostats or other arrays of microbioreactors.

### The Effects of Sample Removal

While the volume of media and the number of cells required for these cellular and molecular analyses are decreasing with the introduction of advanced measurement technologies, there will always be some minimum volume of cells and media required to make the intended measurement. We term this the sample volume. When cells are cultured in commercial-size bioreactors, with volumes of a thousand liters or more, the removal of a 0.5 mL sample for detailed analysis will not perturb the system, but as the volume of the bioreactor is reduced, the fractional perturbation of the withdrawal of a fixed-volume increases as shown in Table 2, which demonstrates that removing a 0.5 mL sample from a 1.0 mL chemostat/bioreactor would represent a 50% reduction in the volume of the media within the chemostat/bioreactor.

TABLE 2
Fractional change in bioreactor volume
upon removal of a 0.5 mL sample

| Reactor volume, mL | 1 | 1.5 | 15 | 250 | 1000 |
|---|---|---|---|---|---|
| Fraction withdrawn | 50% | 33% | 3% | 0.20% | 0.05% |

It is recognized that the repeated removal of samples from an SVL bioreactor will adversely affect bioreactor volume over time, and it can be necessary to consider instantaneous volume rather than the maximum bioreactor volume when determining the concentration and volume of anything that is to be added to the bioreactor.

An obvious alternative to removing a sample from the chemostat/bioreactor would be to accumulate a sufficient sample from the effluent stream and then perform the analysis. The time that this would require depends upon the dilution rate, which might range from 0.01 hr⁻¹ for slowly growing CHO cells to up to ˜0.5 hr⁻¹ for the faster growing yeast Saccharomyces cervisiae, and 0.6 hr⁻¹ for bacteria such as E. coli. As shown in Table 3, the smaller the reactor, the slower the flow rate required to obtain the requisite dilution, and as shown in Table 4, the longer the time required to collect the 0.5 mL sample.

TABLE 3
Flow rate in μL/min for specified dilutions
in chemostats with differing volumes

| | | Chemostat volume, mL: | | | | |
|---|---|---|---|---|---|---|
| | | 1.0 | 1.5 | 15 | 250 | 1000 |
| Dilution | 0.01 | 0.17 | 0.25 | 2.5 | 42 | 170 |
| rate, hr⁻¹ | 0.1 | 1.7 | 2.5 | 25.0 | 420 | 1700 |
| | 0.5 | 8.3 | 13 | 130 | 2,100 | 8,300 |
| | 1 | 17.0 | 25 | 250 | 4,200 | 17,000 |

TABLE 4
Time (min) to collect 0.5 mL of effluent
for differing dilution rates and volumes

Chemostat volume, mL:

| | | 1.0 | 1.5 | 15 | 250 | 1000 |
|---|---|---|---|---|---|---|
| Dilution | 0.01 | 3000 | 2000 | 200 | 12 | 0.03 |
| rate, hr⁻¹ | 0.1 | 300 | 200 | 20 | 1.2 | 0.03 |
| | 0.5 | 60 | 38 | 3.8 | 0.24 | 0.03 |
| | 1 | 29 | 20 | 2.0 | 0.12 | 0.03 |

Tables 2 and 3 illustrate the problem with accumulating measurement-sized samples from low-volume chemostat/bioreactors at low dilution rates: it could take many hours to accumulate the sample, for example 200 minutes to collect a 0.5 mL sample from a 1.5 mL bioreactor operating at a dilution rate of 0.1 hr⁻¹. This poses a significant problem, in that unless all biochemical activity is halted in the collected sample, biochemical reactions will continue in the container as the sample is being accumulated, but the rates and products of these reactions may have no semblance of the rates and products of the reactions occurring within the chemostat because this sample is not being fed fresh media, may have a different composition of dissolved gases, and may have different temperature and stirring/mixing parameters. Simply lysing the cells upon collection will be insufficient, since biochemical reactions will continue even in cell lysate.

Therefore, a heretofore unaddressed need exists in the art to address the aforementioned deficiencies and inadequacies. This invention addresses the issue of sample accumulation, storage, and retrieval from SVL bioreactors, including ones that are operated as chemostats.

### SUMMARY OF THE INVENTION

One aspect of the invention relates to a system of non-perturbative sampling of a sample-volume-limited bioreactor, wherein the removal of a sample from the bioreactor is in a manner that does not perturb the bioreactor's chemical or mechanical microenvironment. The system includes a bioreactor, an input pump, an output pump, and a controller.

The bioreactor comprises a chamber containing media with cells for cell growth and cell division; an input tube coupled to the chamber for delivering at an inflow rate nutrient-laden media that supports the cell growth and the cell division within the chamber; a tube or other means for delivering an input gas mixture into the headspace or the bulk media; a tube or other means for removing the gas from the chamber headspace and/or the bulk media; and an output tube coupled to the chamber for withdrawing a sample of media with cells from the chamber at an outflow rate.

The input pump is coupled to the input tube and configured to operably deliver, by the input tube, the nutrient-laden media at the inflow rate. The output pump is coupled to the output tube and configured to operably withdraw, by the output tube, the sample at the outflow rate.

The controller is coupled to the input pump and the output pump and configured to operate the input pump and the output pump to regulate the inflow rate and the outflow rate, respectively, such that the bioreactor is operable in five modes or phases: a steady-state mode or phase, a sample-accumulation mode or phase, a sample-storage mode or phase, a sample-withdrawal mode or phase, or a volume-restoration mode or phase that returns the bioreactor to it baseline volume, the order of which is under operator control.

In the steady state phase, the density of cells and the level of their metabolic activity in the bioreactor are determined by the media feed rate, the media composition, and cellular genetics and signaling state. Under this invention, the media feed rate is actively controlled, but the media composition is presently assumed fixed. We note that a media formulator at the input of the bioreactor can add and remove specific nutrients and factors from the media that can accelerate or retard cellular division, growth, or metabolic activity, thereby providing an additional means to control cellular activity. Once baseline media composition and flow rate are established and the bioreactor is operating in the steady-state mode, the sample can be accumulated, stored, and then withdrawn and transferred to a collection reservoir; or the sample can be withdrawn from the baseline volume without prior accumulation, with the removed volume subsequently restored. Given a fixed baseline media formulation, the input flow rate determines the cell density. Under this invention, level senses, pump speed, and the height of withdrawal tubes can be used to set the volume of media being perfused during steady-state mode.

During the sample accumulation phase, the media outflow rate is zero or very low and the media inflow rate increases in proportion to an instantaneous volume of media in the chamber so as to accumulate the sample without changes in a metabolic state within the chamber. In the sample storage phase, the inflow and outflow rates of the bioreactor and its mixing and gas exchange rates are matched and are determined by the total volume of the reactor that includes the stored sample, and the mixing and gas exchange rates are appropriately matched to the total bioreactor volume. In the sample withdrawal phase, the outflow rate is significantly higher than the inflow rate to withdraw the accumulated sample from the chamber rapidly at one time. Throughout the withdrawal process, the inflow rate is adjusted to remain in proportion to the instantaneous volume, thereby maintaining a steady chemical state, termed chemostasis, in the chamber. The setting of the rate at which the sample is withdrawn needs to consider the time interval during which the sample is in transit between the bioreactor, whose contents should remain in the microenvironment associated with steady-state chemostasis, and the sample analyzer or sample storage system, where the sample will be denatured or its metabolic processes halted. Typically, this rate might be an order of magnitude or two larger than the volume-defined input flow rate so as to minimize any metabolic or signaling changes in the sample during transit. According to the variable volume approach specified by this invention that at all times fulfills the chemostat condition, it is simply necessary to rapidly decrease the inflow rate as the bioreactor volume is rapidly decreased. In the volume restoration phase, the outflow rate is zero or small, and the input rate is gradually increased in proportion to the instantaneous volume of the bioreactor.

In one embodiment, the bioreactor volume is increased to store a sample for a chosen length of time while maintaining a microenvironment identical to that of the steady-state mode.

In one embodiment, the input pump is a positive displacement pump, or a non-metering pump with an in-line flow sensor including a flow sensor coupled to the input tube and/or a level sensor coupled to the chamber, for controlling the delivery of a predetermined volume of media to the chamber.

In one embodiment, the output pump is a positive displacement pump, or a non-metering pump with an in-line flow sensor including a flow sensor coupled to the output tube, for controlling the withdrawal of a predetermined volume of media and cells from the chamber.

In one embodiment, the system further comprises an additional output pump coupled to an overflow withdrawal tube that is coupled to the chamber and configured to operably set a maximum volume of media in the chamber.

In one embodiment, the controller is further configured to maintain a dilution rate that is a ratio of the inflow rate divided by the instantaneous volume being a constant, so as to maintain a same ratio of nutrient delivery per cell independent of the total volume of the media and the cells that it contains.

In one embodiment, a maximum volume of the sample that is accumulated and then removed is determined by a difference between a maximum allowable volume and a minimum allowable volume of the chamber, and the time required to accumulate the sample is determined by the maximum volume of the sample divided by the inflow rate.

In one embodiment, the inflow rates and the outflow rate are adjustable simultaneously to support different phases of sample accumulation, sample withdrawal, and volume restoration.

In one embodiment, the inflow rate and the outflow rates are of different functions of time such that a difference between the inflow rate and the outflow rate equals a rate of change of the instantaneous volume with time.

In one embodiment, when the inflow rate is greater than the outflow rate, then the volume of media within the chamber increases with time, and when the outflow rate is greater than the inflow rate, the volume of fluid decreases in time.

In one embodiment, the bioreactor is operable in a filling phase during which the outflow rate is zero and the inflow rate is greater than zero, so that the instantaneous volume increases with time, or in a steady-state phase during which the outflow rate is same as the inflow rate.

In one embodiment, the output pump is turned off or otherwise substantially reduced at the beginning of the sample accumulation phase and turned on or otherwise substantially increased at the beginning of the sample withdrawal phase.

In one embodiment, the bioreactor further comprises a means for stirring/mixing and oxygenating the media in the chamber respectively at a stirring/mixing rate and a gas exchange rate to ensure that the media within the chamber is well mixed, uniformly oxygenated, and at a desired pH over a full range of volumes of media contained in the chamber during all phases of operation.

In one embodiment, the stirring/mixing rate, the gas exchange rate, and an input gas mixture composition are adjustable so as to ensure that local conditions throughout the chamber remain unchanged over the full range of volumes of media contained in the bioreactor chamber during all phases of operation.

In one embodiment, growth conditions, and nutrient and gas concentrations within the entire media in the chamber are maintained at baseline conditions by modulating the inflow rate, the outflow rate, the gas exchange rate and the stirring/mixing rate in a manner that maintains static biochemical conditions independent of the instantaneous volume of media and cells within the chamber.

In one embodiment, a fraction of the media in the chamber is withdrawable without prior accumulation, but with the inflow rate, the outflow rate, the gas exchange rate and the stirring/mixing rate modulated post-withdrawal in a manner that maintains static biochemical conditions independent of the instantaneous volume of cells and media within the chamber.

In one embodiment, by the dynamic control of the inflow rate, the outflow rate, the gas exchange rate, and the stirring/mixing rate, any arbitrary volume within the chamber can be maintained at the same biochemical state as any volume within an industry-standard, constant-volume chemostat is achievable.

In one embodiment, the gas is delivered and exchanged through the headspace.

In one embodiment, the gas is delivered through a sparger immersed in the fluid.

In one embodiment, the gas is delivered and exchanged using a membrane displacement/gas exchanger/mixer.

In one embodiment, the media is mixed using a stir bar.

In one embodiment, the media and cells are mixed with an impeller or propeller.

In one embodiment, the media and cells are mixed using a membrane displacement/gas exchanger/mixer.

In another aspect, the invention relates to a method for operating a chemostat/bioreactor with a variable volume, wherein the variable-volume chemostat/bioreactor is characterized with a media volume that varies with time in a chamber, an inflow rate at which nutrient-laden media is delivered into the chemostat/bioreactor such that the chemostat/bioreactor can support the desired cell density, with the result that the media composition within the bioreactor stays constant over time, and an outflow rate at which a sample is withdrawn from the chemostat/bioreactor that is determined by the maximum allowable interval of time between initiation and completion of sample withdrawal so as to minimize metabolic changes in the sample during transit. The method comprises regulating the inflow rate, the outflow rate, and the input gas mixture such that the chemostat/bioreactor operates in a volume restoration phase immediately following the sample withdrawal/transfer phase, wherein for the sample withdrawal phase, the outflow rate is higher than the inflow rate to withdraw the sample volume from the chamber rapidly at one time and the inflow rate and gas exchange rate remain in proportion to the instantaneous volume to maintain chemostasis in the chamber; wherein for the volume restoration phase, the outflow rate is zero or very small and the inflow rate and gas exchange rate increase in proportion to the instantaneous volume of media in the chamber so as to restore the volume without changes in a metabolic state within the chamber.

In one embodiment, a dilution rate that is a ratio of the inflow rate divided by the instantaneous volume is a constant, so as to maintain the same ratio of nutrient delivery per cell independent of the total volume of the media and cells.

In one embodiment, the method further comprises stirring/mixing and oxygenating the media in the chamber respectfully at a stirring/mixing rate and a gas exchange rate to ensure that the media within the chamber is well mixed, uniformly oxygenated, and has the desired carbon dioxide levels.

In one embodiment, the gas exchange rate and the stirring/mixing rate are adjustable so as to ensure that the local conditions throughout the bioreactor chamber remain unchanged.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A-1C show schematically a yeast chemostat with input and output pumps, gas mixture delivery, gas venting, and a stirring/mixing means according to embodiments of the invention. In operation as a classical chemostat (FIG. 1A), the input and output flow rates are equal so that the volume is constant over time. Together FIGS. 1A-1C show schematic representations of three time points in the operation of a variable-volume chemostat/bioreactor. A) Steady-state phase; B) End of sample accumulation phase; and C) End of sample withdrawal phase. Note that the media and gas flow rates in (B) are twice those in (A), as would be appropriate with the media volume in (B) being twice that in (A). In (C), the output flow is transiently very high as the input and gas flows return to their baseline values, consistent with bioreactor volume approaching the maximum volume of the bioreactor. Operating as a variable-volume chemostat/bioreactor, the instantaneous input flow rate would be determined by the volume at each moment of time during the accumulation (FIG. 1B) and removal (FIG. 1C) phases.

FIGS. 2A-2C show a different embodiment of the device in FIGS. 1A-1C, in that there are two output pumps. FIG. 2A shows the situation during the sample accumulation phase, with both output pumps off and the level of fluid in the reactor rising. FIG. 2B shows the bioreactor at the end of the sample accumulation phase, where output pump 2 and the overflow withdrawal tube remove excess media so that the bioreactor can even operate in the steady state with a high, fixed fluid level, with the height of the open end of the sample withdrawal tube for output pump 2 setting the maximum level and the bioreactor output being delivered to an overflow reservoir.

FIG. 2C shows the end of the sample withdrawal phase, where the lower height of the open end of the sample withdrawal tube for output pump 1 determines the minimum level of fluid in the reactor when withdrawing the sample from the reactor or operating in the steady state with a low, fixed fluid level.

FIGS. 3A-3B show respectively (FIG. 3A) the media and gas mixture formulators, level and flow sensors, fluid lines, and the master controller required to fully automate the cell culture process, and (FIG. 3B) the system showing the sense and control lines that connect the master controller to the sensors and pumps.

FIG. 4A shows schematically the time-sequence of the bioreactor volume and the rates of media delivery (Qᵢ), sample accumulation (Vol), and sample withdrawal (Qo) in a variable-volume chemostat/bioreactor according to embodiments of the invention, with the sample being replaced upon withdrawal from a filled bioreactor (B-E), or the bioreactor being overfilled before sample withdrawal (E-H). A) Filling phase: No withdrawal; the volume increases exponentially with time to maintain chemostat conditions. B) Steady-state chemostat phase: The delivery rate equals the withdrawal rate. C) Sample withdrawal phase: The sample is withdrawn at a constant rate, and the delivery rate decreases as the volume of the bioreactor is decreased, thereby also maintaining the chemostat conditions. D) Sample accumulation phase: No withdrawal; the delivery rate increases in proportion to volume to maintain chemical steady state, with slight exponential curvature evident in volume. E) Steady-state chemostat phase: The delivery rate equals the withdrawal rate. F) Sample accumulation phase: The input flow rate increases with the bioreactor volume, and there is no output. G) Sample withdrawal phase: Accelerated sample withdrawal; media delivery remains in proportion to the volume to maintain chemostasis, with an almost undetectable curvature. H) Steady-state chemostat phase. The gas mixture and stirring/mixing rate would be regulated as necessary during all phases.

FIG. 4B shows schematically the time-sequence of the bioreactor volume and the rates of media delivery (Qᵢ), sample accumulation (Vol), and sample withdrawal (Qo) in a variable-volume chemostat/bioreactor according to embodiments of the invention, with the sample being stored in the fully filled bioreactor (D) prior to withdrawal (E). A) Filling phase: No withdrawal; the volume increases linearly with time to maintain chemostat conditions. B) Steady-state chemostat phase: The delivery rate equals the withdrawal rate. C) Sample accumulation phase: The input flow rates increase with bioreactor volume, and there is no output. D) Sample storage phase, with appropriately increased and identical input and output pump rates. E) Sample withdrawal phase: Accelerated sample withdrawal, media delivery remains in proportion to volume to maintain chemostasis, with an evident curvature. F) Steady-state chemostat phase. The gas mixture and stirring/mixing rate would be regulated as necessary during all phases.

FIG. 5 shows schematically a sequence of events similar to that of FIG. 4A, except that the initial volume increase is linear with time, thereby not satisfying the chemostat condition, but with two accumulation and withdrawal phases that do satisfy that condition.

FIGS. 6A-6D show schematically four controller schemes, two with positive displacement pumps (FIG. 6A and FIG. 6B) that can increment or decrement the volume-tracking totalizer within the controller, and two that use centrifugal pumps (FIG. 6C and FIG. 6D), which do not have a direct relation between volume moved and motor rotations but have integral flow sensors that inform the totalizer as to how much fluid is moved into or out of the bioreactor. Two systems do not have bioreactor level sensors (FIG. 6A and FIG. 6C), while two do (FIG. 6B and FIG. 6D). In all cases, gas delivery and removal and stirring/mixing are controlled by the microcontroller.

FIG. 7 shows schematically a system with a third pump and sample/split-flow controller that are used to transfer sample or bulk media with cells to a second reactor vessel via a second output fluid sink to initiate another series of cultures.

FIG. 8 shows schematically a flow chart for the control sequence to be implemented by the system in FIGS. 3A-3B, 6A-6D, and 7 to restore bioreactor volume after transfer of a sample to another reservoir for off-line analysis or seeding another culture as shown in FIG. 4A intervals C and D.

FIG. 9 shows schematically a flow chart for the control sequence to be implemented by the system in FIGS. 3A-3B, 6A-6D, and 7 to accumulate a sample in a bioreactor prior to its transfer to another reservoir for off-line analysis or seeding another culture as shown in FIG. 4A intervals F and G and FIG. 4B intervals C-E.

FIGS. 10A-10B show schematically two methods for mixing and gas exchange in SVL bioreactors. FIG. 10A: The use of a magnetic or shaft-driven stir bar for mixing, and headspace gas exchange and gas sparging in the liquid for controlling the levels of dissolved gas in the media. FIG. 10B: The use of a displacement gas exchanger/mixer and headspace gas exchange to mix and control media gas levels. The vertical motion of the displacement gas exchanger/mixer provides efficient vertical mixing of a deep well, and continuously exposes the oxygen- and carbon dioxide permeable gas exchange membrane to media such that oxygen rapidly diffuses out of the bulb and carbon dioxide rapidly diffuses into the bulb. Control of the partial pressure of carbon dioxide in the bulb can also be used to adjust the pH of the bioreactor.

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially” or “approximately” shall generally mean within 20 percent, preferably within 10 percent, and more preferably within 5 percent of a given value or range. Numerical quantities given herein are approximate, meaning that the terms “around,” “about,” “substantially” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. As used herein, the term “and/or” includes any and all combinations of one or more of the associated listed items.

As used herein, the term “chemostat” generally refers to a constant volume bioreactor with matched input and output media pump rates, and a “variable-volume chemostat/bioreactor” or a “variable-volume chemostat” or a “variable-volume bioreactor” generally refers to a bioreactor where the input flow rate is adjusted to match the instantaneous volume of the media in the bioreactor. However, since a chemostat is a form of bioreactor, there may be cases where this terminology is not adhered to exactly.

As used herein, the term “sample-volume-limited bioreactor” refers to any bioreactor for which the intended sample volume represents a larger than acceptable percentage of the bioreactor volume as related to how the addition of media or the removal of cells and media might affect the metabolic activity of the cells within the bioreactor.

As used herein, the term “stirring/mixing” refers to any of the many ways in which the contents of a bioreactor can be homogenized, for example by stirring that is driven by a rotating paddle, propeller, or stir bar, or by a vertically oscillating membrane displacement gas exchanger/mixer that displaces liquid and sheds vortices that mix the fluid. This invention does not depend upon the choice of a particular stirring/mixing mechanism.

It will be understood that the terms “gas delivery” and “gas and pH regulation” will comprise the regulatory steps required to maintain constant concentrations of dissolved oxygen and carbon dioxide and a constant pH in the media as the volume of media and total number of cells in the bioreactor are changed. In all discussions of the operation of a chemostat/bioreactor in variable-volume mode, it will be assumed in every instance that gas and pH regulation can be applied whenever appropriate, and hereafter will not be addressed specifically. The term “gas exchange” will refer to the diffusive or convective flow of gas across the gas-permeable membrane of the membrane displacement gas exchanger/mixer.

As used herein, the terms “controller,” “master controller,” and “microcontroller” are interchangeable and refer at the minimum to a small computer on a single integrated circuit containing one or more CPUs along with memory and programmable input/output peripherals, or a larger computer or programmable control system.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

The cost of cells, media, hardware, physical infrastructure, and staff required to operate intermediate-sized (2 mL to 250 mL) to large (1 L to 10,000 L) bioreactors severely limits their utility in the discovery, engineering, and optimization of cells that produce a particular biopharmaceutical or other chemical substance. There is a clear need to operate large numbers of smaller bioreactors to enable rapid and cost-effective screening of microbes and mammalian cells used in the manufacture of biochemicals, pharmaceuticals, and other chemical entities and to optimize the media used to culture these cells. It is often necessary to regularly withdraw cell and media samples from small bioreactors to characterize the gene expression and metabolic state of the cells and determine whether they are operating within the intended performance envelope. As these bioreactors are made with smaller and smaller volumes, it becomes increasingly difficult to remove samples from these bioreactors, in that the removal of the sample and the possible restoration of the sample volume with fresh media will perturb or otherwise disrupt the metabolic activity and gene expression profiles of the cells being cultured. In recognition of the challenges of removing samples from a small bioreactor without disturbing the remaining cells, we introduce the concept of a sample-volume-limited (SVL) bioreactor for which sample withdrawal is problematic.

As an example of a system for which this is a particular problem, chemostats are a type of continuous-flow bioreactors used to grow and study prokaryotic microbes such as E. coli, yeast, or suspended mammalian or other eukaryotic cells grown in suspension. This invention provides a novel approach to remove samples from an SVL chemostat or other small bioreactors in a manner that does not disrupt the biochemical equilibrium within the chemostat. In a conventional chemostat, a continuous flow of media into the bioreactor is matched by an equal and opposite flow of conditioned media and cells out of the bioreactor, so that the volume of fluid in the bioreactor is a constant. As cells grow and divide, some of the cells are washed out. Unless the flow rates are so high as to wash all the cells from the bioreactor, the system will reach a steady state determined by both the flow rate and the concentration of nutrients in the input stream, and the chemical conditions and number of cells in the chemostat at any time will remain constant, hence the term “chemostat,” with “chemo” referring to the biochemistry within the reactor and “stat” referring to “static.” The effluent from a chemostat can be collected for analysis or transfer to another bioreactor for subsequent culture or population expansion. As the chemostats are made smaller, the flow rate required to maintain the smaller number of cells is reduced correspondingly, thereby extending the time required to collect a specific volume for analysis.

However, the biochemical processes occurring within the bioreactor might continue in the sample collection portion of the apparatus, which has different growth conditions and nutrient and gas concentrations. The ongoing biochemistry in the collection portion of the apparatus could be slowed or halted by immediately freezing the sample or heating it to a point where enzymes are degraded, both of which complicate the sample collection process, particularly when it is desired to operate a large number of chemostats in parallel. A similar problem occurs when a significant fraction of the volume of a chemostat or other bioreactor is needed to inoculate another culture system to create a replicate culture, in that removal of a fraction of the media and cells from the first chemostat or bioreactor could lead to altered biochemical conditions for the remaining cells, which might be needed to grow more cells to seed additional bioreactors.

There are two well-known solutions to this problem of biochemical changes with a sample after collection: stop protein biochemistry by flash heating the sample as it is being collected, with or without lysing, to denature proteins, or freeze it to approximately −80° C. The limitations of the former are that not all biochemical reactions within cell media or lysate are driven by enzymes or other proteins and will continue even after protein denaturation, and it may be difficult to properly denature proteins without producing peptide fragments or altering other biomolecules. The limitations of the latter solution are that biochemistry will resume as the sample is being thawed for analysis, and it may be difficult to simultaneously thaw the entire sample. In either case, the problems in making biochemical measurements on samples that are collected over time will limit the ability to quantify short-lived or labile species in the sample.

In view of the aforementioned deficiencies and inadequacies, this invention provides a novel approach: the media (fluid) in the bioreactor is allowed to accumulate in the bioreactor portion of the apparatus by slowing or stopping the media removal rate. The excess volume constitutes the sample that is to be removed from the bioreactor rapidly at one time. The speed with which the sample is removed would be determined by the acceptable level of metabolic changes that might occur during the transfer of the sample. The growth conditions and nutrient and gas concentrations within the entire volume in the bioreactor are maintained at the original conditions by modulating the input flow (or inflow) rate, the output flow (or outflow) rate, the gas exchange rates, and the stirring/mixing rate in a manner that maintains static biochemical conditions independent of the instantaneous volume of cells and media within the bioreactor vessel or chamber. Similarly, a substantial fraction of a bioreactor could be removed without prior accumulation, but with the input flow, output flow, gas exchange, and stirring/mixing rates modulated post-withdrawal in a manner that maintains static biochemical conditions independent of the instantaneous volume of cells and media within the bioreactor chamber. The variable-volume phase of bioreactor operation could occur before or after sample removal, depending upon whether the bioreactor had any available excess volume or could support growth from a partially filled bioreactor. The input and output flow rates can both be modulated simultaneously to support different durations of sample removal and volume restoration. Hence the dynamic control of bioreactor parameters such as the rates of input flow (or inflow), output flow (or outflow), gas exchange, and stirring/mixing can hold any arbitrary volume within the bioreactor chamber at the same biochemical state as any volume within an industry-standard, constant-volume bioreactor, as would be required to harvest a substantial fraction of a bioreactor's volume for either sample analysis or the seeding of another bioreactor.

While one might view a “variable-volume chemostat” as an oxymoron, it is important to realize that the key property of a conventional chemostat is that it holds all of the cells it contains in a biochemical steady state, hence the name. The uniform biochemical state of a conventional, well-mixed chemostat implies that each incremental volume element within that chemostat is in the same steady chemical state, i.e., a steady microenvironment. Just as density is an intrinsic variable describing mass per unit volume and the mass of an object is an extrinsic variable representing the integral of the density over the entire volume of an object, we make the distinction of an “intrinsic chemostasis” and “extrinsic chemostasis.” Within a variable-volume chemostat/bioreactor, each elemental volume enjoys an identical intrinsic chemostasis, but the chemostat/bioreactor in its entirety is not static since its volume and the number of elemental volumes within it change over time. Hence it is appropriate to describe a variable-volume chemostat/bioreactor as an intrinsic chemostat. In contrast, a classical, fixed-volume chemostat exhibits both intrinsic and extrinsic chemostasis. Fed-batch bioreactors share many of the properties of chemostats, in that in certain operational modes they are perfused continuously and their volume may increase in time until the cells and media are harvested. Continuous perfusion bioreactors also share many features with classical chemostats, with the exception that cells are actively restrained from leaving the bioreactor or are captured, separated from the effluent media, and returned to undergo subsequent division and product production.

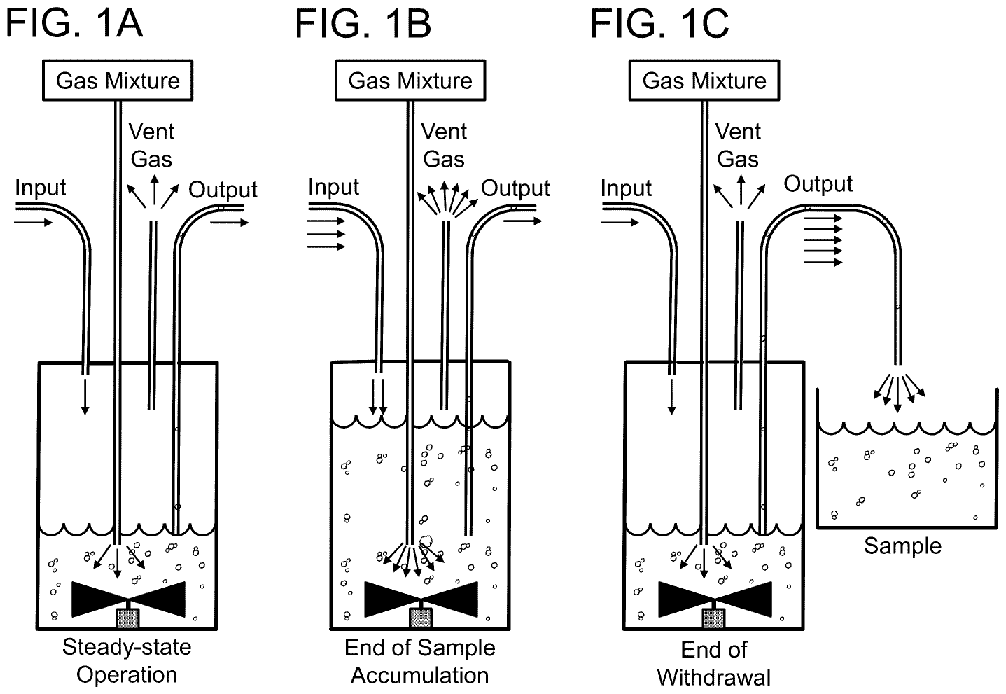

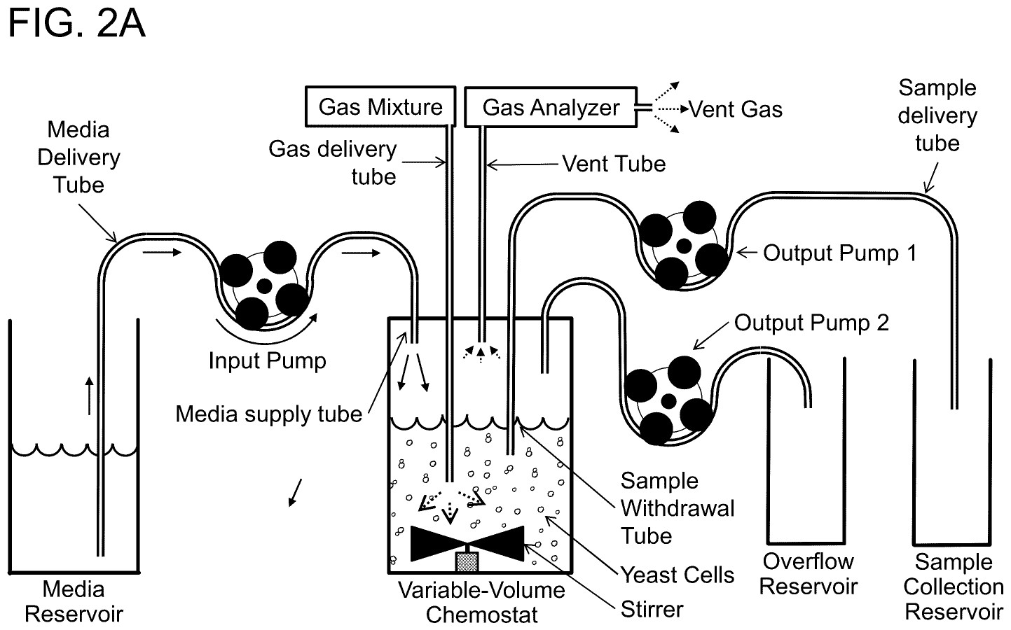

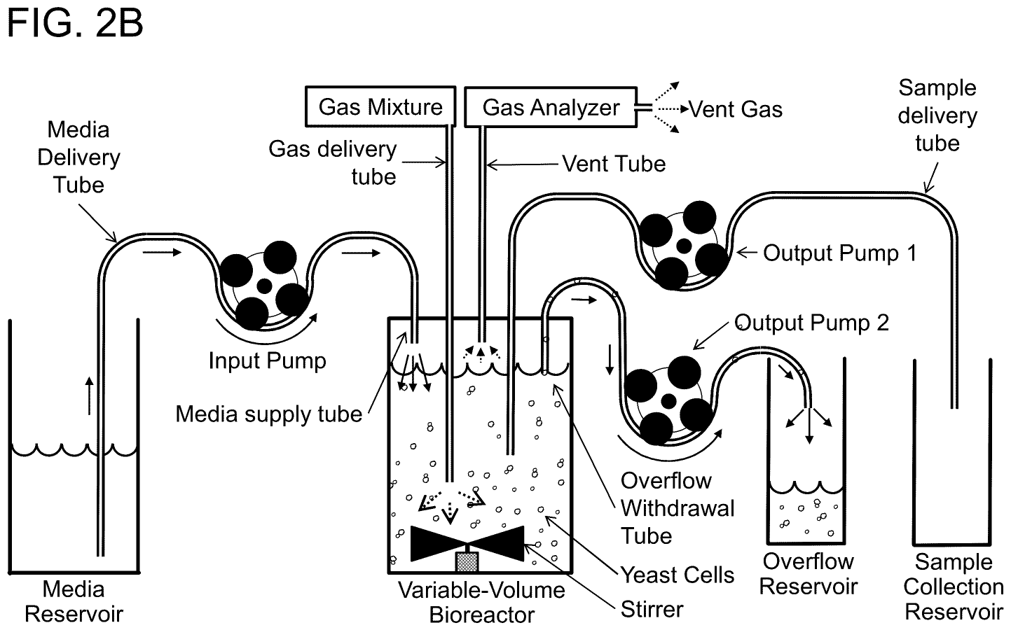

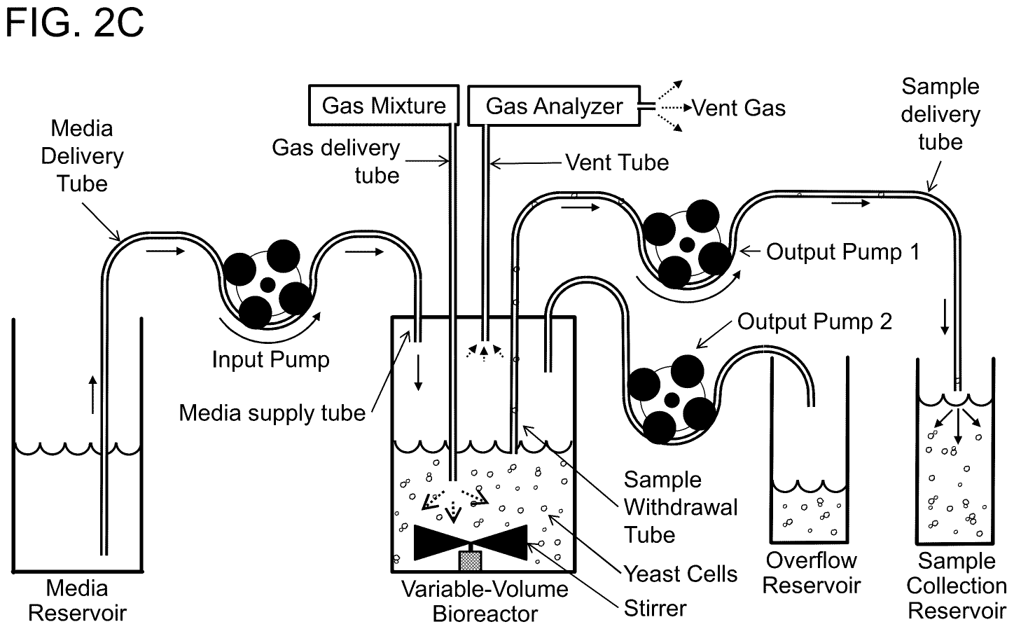

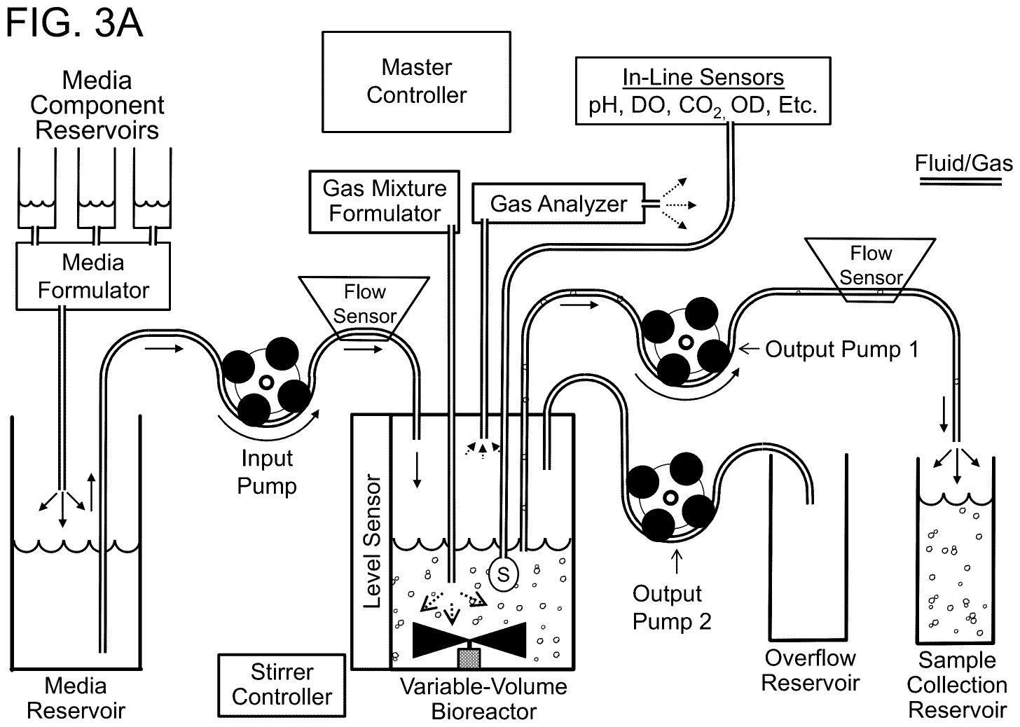

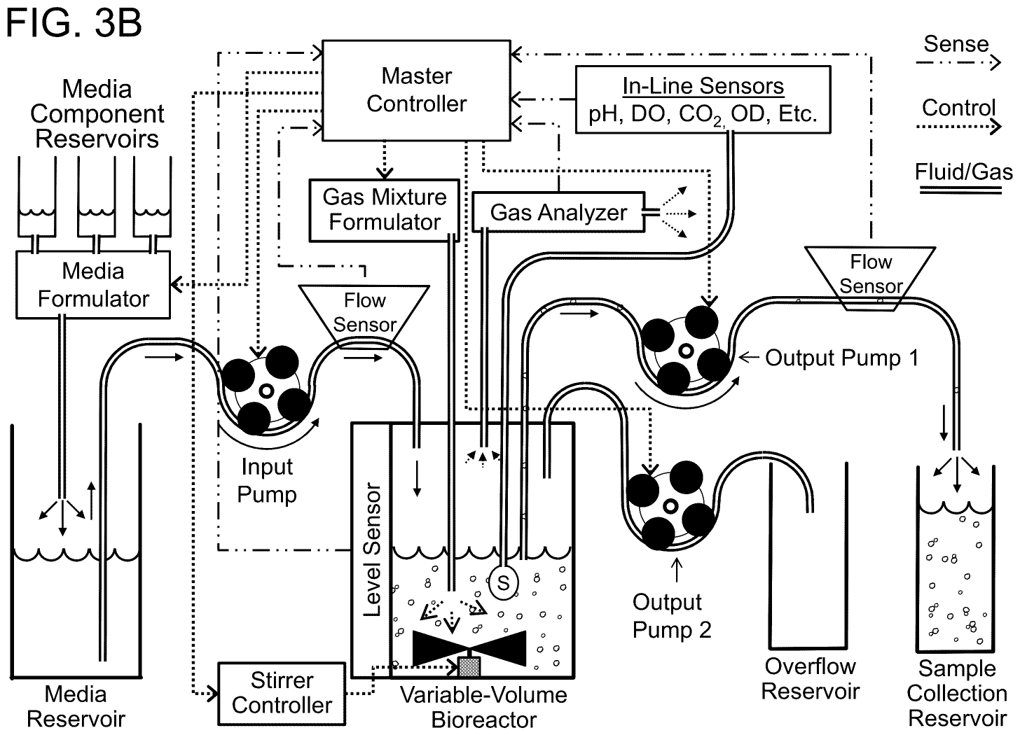

Referring to FIGS. 1A-1C, the bioreactor comprises a chamber containing media with cells for cell growth and cell division; an input tube coupled to the chamber for delivering nutrient-laden media that supports the cell growth and the cell division within the chamber, at an inflow rate; and an output tube coupled to the chamber for withdrawing a sample from the chamber at an outflow rate. As illustrated schematically in FIGS. 2A-2C and FIGS. 3A-3B, the inflow rate and the outflow rate are regulated, for example, by a master controller that controls the operations of an input pump and an output pump in terms of pump speed and run time, such that the bioreactor is operable in a sample accumulation phase or a sample withdrawal phase immediately following the sample accumulation phase. In the sample accumulation phase, the outflow rate is zero and the inflow rate increases in proportion to the instantaneous volume of media in the chamber so as to accumulate the sample without changes in a metabolic state within the chamber. In the sample storage phase, the input and output rates are equal and set in proportion to the increased bioreactor volume so as to satisfy the baseline steady-state chemostat condition. In the sample withdrawal phase, the outflow rate is regulated at a higher rate than the inflow rate to withdraw the accumulated sample from the chamber rapidly at one time and the inflow rate remains in proportion to the instantaneous volume to maintain chemostasis in the chamber. As discussed previously, depending on the operational protocol, gas and pH may need to be regulated during these phases.

Without intent to limit the scope of the invention, examples according to the embodiments of the invention are given below.

In some embodiments, as shown in FIGS. 3A-3B, 6A-6D and 7, the system for non-perturbative sampling of SVL bioreactors comprises a bioreactor, an input pump, an output pump, and a controller. The bioreactor comprises a bioreactor chamber containing media with cells for cell maintenance, growth, and division; an input tube coupled to the chamber for delivering nutrient-laden media that supports the cell maintenance, growth, and division within the chamber; and an output tube coupled to the chamber for withdrawing a sample from the chamber. The input pump is coupled to the input tube and configured to operably deliver, by the input tube, the nutrient-laden media at an inflow rate. The output pump is coupled to the output tube and configured to operably withdraw, by the output tube, the sample at an outflow rate. The controller is coupled to the input pump and the output pump and configured to operate the input pump and the output pump to regulate the inflow rate and the outflow rate, respectively, such that the chemostat/bioreactor is a variable-volume bioreactor in which an instantaneous volume of media either varies continuously with time or is held constant, and operable in a sample accumulation phase, a sample withdrawal phase, or a volume restoration phase, wherein the sample withdrawal phase either follows the sample accumulation phase or precedes the volume restoration phase, wherein the inflow rate increases in proportion to an increase of the instantaneous volume of media in the chamber in the sample accumulation phase and decreases in proportion to a decrease of the instantaneous volume of the chamber in the sample withdrawal phase, so as to maintain the quantity of nutrients per cell within the media in the chamber unchanged.

In some embodiments, the input pump is a positive displacement pump, or a non-metering pump with an in-line flow sensor including a flow sensor coupled to the input tube and/or a level sensor coupled to the chamber, for controlling the delivery of a predetermined volume of media to the chamber.

In some embodiments, the output pump is a positive displacement pump, or a non-metering pump with an in-line flow sensor including a flow sensor coupled to the output tube, for controlling the withdrawal of a predetermined volume of media and cells from the chamber.

In some embodiments, the system further comprises an additional output pump coupled to an overflow withdrawal tube that is coupled to the chamber and configured to operably set a maximum volume of media in the chamber.

In some embodiments, the controller is further configured to maintain a dilution rate that is a ratio of the inflow rate divided by the instantaneous volume being a constant, so as to maintain a same ratio of nutrient delivery per cell independent of the total volume of the media and the cells that it contains.

In some embodiments, a maximum volume of the sample that is accumulated and then removed is determined by a difference between a maximum allowable volume and a minimum allowable volume of the chamber, and the time required to accumulate the sample is determined by the maximum volume of the sample divided by the inflow rate.

In some embodiments, the inflow rates and the outflow rate are adjustable simultaneously to support different phases of sample accumulation, sample withdrawal, and volume restoration.

In some embodiments, the inflow rate and the outflow rates are of different functions of time such that a difference between the inflow rate and the outflow rate equals a rate of change of the instantaneous volume with time.

In some embodiments, when the inflow rate is greater than the outflow rate, then the volume of media within the chamber increases with time, and when the outflow rate is greater than the inflow rate, the volume of fluid decreases in time.

In some embodiments, the bioreactor is operable in a filling phase during which the outflow rate is zero and the inflow rate is greater than zero so that the instantaneous volume increases with time, or in a steady-state phase during which the outflow rate is same as the inflow rate.

In some embodiments, the output pump is turned off or otherwise substantially reduced at the beginning of the sample accumulation phase and turned on or otherwise substantially increased at the beginning of the sample withdrawal phase.

In some embodiments, the bioreactor further comprises a means for stirring/mixing and oxygenating the media in the chamber respectively at a stirring/mixing rate and a gas exchange rate to ensure that the media within the chamber is well mixed, uniformly oxygenated, and at a desired pH over a full range of volumes of media contained in the chamber during all phases of operation.

In some embodiments, the stirring/mixing rate, the gas exchange rate, and an input gas mixture composition are adjustable so as to ensure that local conditions throughout the chamber remain unchanged over the full range of volumes of media contained in the bioreactor chamber during all phases of operation.

In some embodiments, growth conditions and nutrient and gas concentrations within the entire media in the chamber are maintained at original conditions by modulating the inflow rate, the outflow rate, the gas exchange rate, and the stirring/mixing rate in a manner that maintains static biochemical conditions independent of the instantaneous volume of media and cells within the chamber.

In some embodiments, a fraction of the media in the chamber is withdrawable without prior accumulation, but with the inflow rate, the outflow rate, the gas exchange rate, and the stirring/mixing rate modulated post-withdrawal in a manner that maintains static biochemical conditions independent of the instantaneous volume of cells and media within the chamber.

In some embodiments, by the dynamic control of the inflow rate, the outflow rate, the gas exchange rate, and the stirring/mixing rate, any arbitrary volume within the chamber can be maintained at the same biochemical state as any volume within an industry-standard, constant-volume chemostat/bioreactor is achievable.

In some embodiments, the gas is delivered and exchanged through the headspace.

In some embodiments, the gas is delivered through a sparger immersed in the fluid.

In some embodiments, the gas is delivered and exchanged using a membrane displacement gas exchanger/mixer.

In some embodiments, the media is mixed using a stir bar.

In some embodiments, the media and cells are mixed with an impeller or propeller.

In some embodiments, the media and cells are mixed using a membrane displacement gas exchanger/mixer.

In some embodiments, the method is disclosed for operating a bioreactor with a variable volume, wherein the variable-volume bioreactor is characterized with a media volume that varies with time in a chamber, an inflow rate at which nutrient-laden media is delivered into the bioreactor, such that the media composition stays constant over time, and an outflow rate at which a sample is withdrawn from the bioreactor.

The method comprises regulating the inflow rate, the outflow rate, and the input gas mixture such that the bioreactor operates in a volume restoration phase immediately following the sample withdrawal/transfer phase, wherein for the sample withdrawal phase, the outflow rate higher than the inflow rate to withdraw the sample volume from the chamber rapidly at one time and the inflow rate and gas exchange rate remain in proportion to the instantaneous volume to maintain chemostasis in the chamber; wherein for the volume restoration phase, the outflow rate is zero or very small and the inflow rate and gas exchange rate increase in proportion to the instantaneous volume of media in the chamber so as to restore the volume without changes in a metabolic state within the chamber.

In some embodiments, a dilution rate that is a ratio of the inflow rate divided by the instantaneous volume is a constant, so as to maintain the same ratio of nutrient delivery per cell independent of the total volume of the media and cells.

In some embodiments, the method further comprises stirring/mixing and oxygenating the media in the chamber respectfully at a stirring/mixing rate and a gas exchange rate to ensure that the media within the chamber is well mixed and uniformly oxygenated and has the desired carbon dioxide levels.

In some embodiments, the gas exchange rate and the stirring/mixing rate are adjustable so as to ensure that the local conditions throughout the bioreactor chamber remain unchanged.

In addition, in some embodiments, whether the chemostat/bioreactor is being operated in an aerobic or anaerobic mode, the values of other cell culture parameters would determine whether it is necessary to adjust over time buffer concentrations in the input media and the partial pressures of oxygen, carbon dioxide, and nitrogen in the incoming gas mixtures to maintain constant concentrations of dissolved oxygen and carbon dioxide and a constant pH in the media as the volume of media and total number of cells in the bioreactor are changed. In some embodiments, this process is referred to as gas and pH regulation.

In some embodiments, the method may also include the regulatory steps for maintaining constant concentrations of dissolved oxygen and carbon dioxide and a constant pH in the media as the volume of media and total number of cells in the bioreactor are changed. In all discussions of the operation of a chemostat/bioreactor in variable-volume mode, it is assumed in every instance that gas and pH regulation can be applied whenever appropriate, and hereafter will not be addressed specifically.

These and other aspects of the invention are further described below.

As disclosed above, the novel approach of the invention is not to move the sample to a separate container for accumulation and subsequent analysis, but to store the sample within the bioreactor where the entire sample and the other cells in the bioreactor will enjoy the same conditions while the sample is being accumulated. However, it is the unquestioned dogma that chemostats have constant volume, achieved by having equal inflow and outflow rates. Relaxing the constant volume constraint in fact solves the accumulation problem, as long as the local conditions within the chemostat/bioreactor are unchanged as its volume increases. Suppose that the output pump is turned off at the beginning of the sample accumulation period, so that the volume increases linearly with time, as determined by a fixed input flow rate. During this time, the number of cells in the chemostat/bioreactor will increase, since cells are no longer being removed. There will be an increase in the total amount of metabolites in the bioreactor, but because new media is being added at a rate proportional to the bioreactor volume, the concentration of these metabolites will remain constant if the cell metabolic rates are unchanged and the media inflow remains constant. However, if the inflow rate is held constant during sample accumulation, the increase in chemostat/bioreactor volume will mean that the nutrients being delivered will be shared by the ever-growing number of cells, for a net decrease in available nutrients per cell. This in turn will reduce the rate at which metabolites are produced. This means that simply turning off the output pump is an inadequate means for accumulating a sample that is also representative of the steady-state phase that preceded sample accumulation.

Hence, under a variable-volume protocol, the flow rate must be increased in proportion to the increase in volume so that the nutrients available per cell will remain unchanged. Appropriate changes in gas exchange and stirring/mixing will ensure that the local conditions throughout the bioreactor remain unchanged. In the standard, constant-volume chemostat, the effluent immediately after its removal is only initially in equilibrium with the bulk media in the chemostat, and that equilibrium is quickly lost as the metabolically active cells are confined to the removal tubing or a passive collection reservoir where fresh nutrients are no longer being delivered to cells, gas is no longer being exchanged, and the only stirring/mixing in the tube comes from the shear force applied by viscous drag against the tubing wall, and the collection reservoir may not be stirred. In the variable-volume chemostat/bioreactor, the accumulated sample remains in equilibrium within the chemostat/bioreactor, with the same nutrient delivery rate per cell, the same gas exchange, and the same stirring/mixing forces. In other words, the variable-volume chemostat/bioreactor stores the accumulating sample within itself. This feature is not needed for large chemostats, where the removal of a sample does not appreciably affect the remaining chemostat volume. In contrast, the removal of a small sample from an only slightly larger classic chemostat would be metabolically disruptive to the chemostat. Similar considerations will apply to small variable-volume fed-batch bioreactors.

FIGS. 2A-2C show schematic representations of three time points in the operation of a variable-volume bioreactor that can be operated as a variable-volume chemostat/bioreactor. FIG. 2A shows the situation midway through the sample accumulation phase, with both output pumps off and the level of fluid in the reactor rising. FIG. 2B shows the bioreactor at the end of the sample accumulation phase, where output pump 2 and the overflow withdrawal tube remove excess media so that the bioreactor can operate with a fixed, elevated fluid level, with the height of the open end of the sample withdrawal tube for output pump 2 setting the maximum level and the bioreactor output being delivered to an overflow reservoir. FIG. 2C shows the end of the sample withdrawal phase, where the height of the open end of the sample withdrawal tube for output pump 1 determines the minimum level of fluid in the reactor when withdrawing the sample from the reactor. Note that the input flow is higher in FIG. 2C than in FIG. 2B, which in turn is higher than that in FIG. 2A, as appropriate for the fact that the bioreactor volume in FIG. 2C is greater than that in FIG. 2B, which is greater than that in FIG. 2A. Afterwards, the input pump rates, mixing speed, and gas exchange rates are returned to their baseline values, consistent with bioreactor volume approaching the steady-state value. The gas mixture and exchange rate would be regulated as necessary during all stages of sample accumulation and delivery. It is important to recognize that at the three instances shown, the metabolic state of all the cells shown is identical, even those in the just-removed sample, hence the merit of the variable-volume chemostat/bioreactor approach.

FIGS. 3A-3B show schematically a control system that can be applied to the bioreactor in FIGS. 2A-2C. The key components for sensing and control are a master controller unit, a set of input media reservoirs and a media formulator, a gas mixture formulator, a gas analyzer, and an input pump that is either a positive displacement pump that can deliver specified volumes of media to the bioreactor, or a centrifugal or other pump for which there is not a well-defined relationship between a pump rotation angle and a volume delivered, which requires the use of an in-line flow sensor and/or the use of a level sensor in the bioreactor. Not shown is an electronic scale that weighs the bioreactor and can be used as a level sensor. The bioreactor has a controller for the variable speed of the stirrer, and a sensor system S that measures pH, dissolved oxygen (DO) and carbon dioxide, optical density (OD) or cell density and viability, and lactate, glucose, and ammonia concentrations, among other biochemical variables. One output pump (Output Pump 1), again with cither positive displacement or non-metering with an in-line flow sensor, controls the removal of a known volume of media and cells from the bioreactor. A second output pump (Output Pump 2) connected to the overflow withdrawal tube can be used to set the maximum volume of fluid in the bioreactor. These individual units are interconnected by gas and fluid tubing or fluidic circuits. FIG. 3B shows how the level, flow, and chemical sensors are connected to the master controller by electrical sense lines, and how the master controller is connected to the formulators and pumps by electrical control lines. Together, this system can perform non-perturbative sampling of SVL bioreactors. The system shown in FIGS. 3A-3B is just one embodiment of such a system, and other variations could support the same requisite functions.

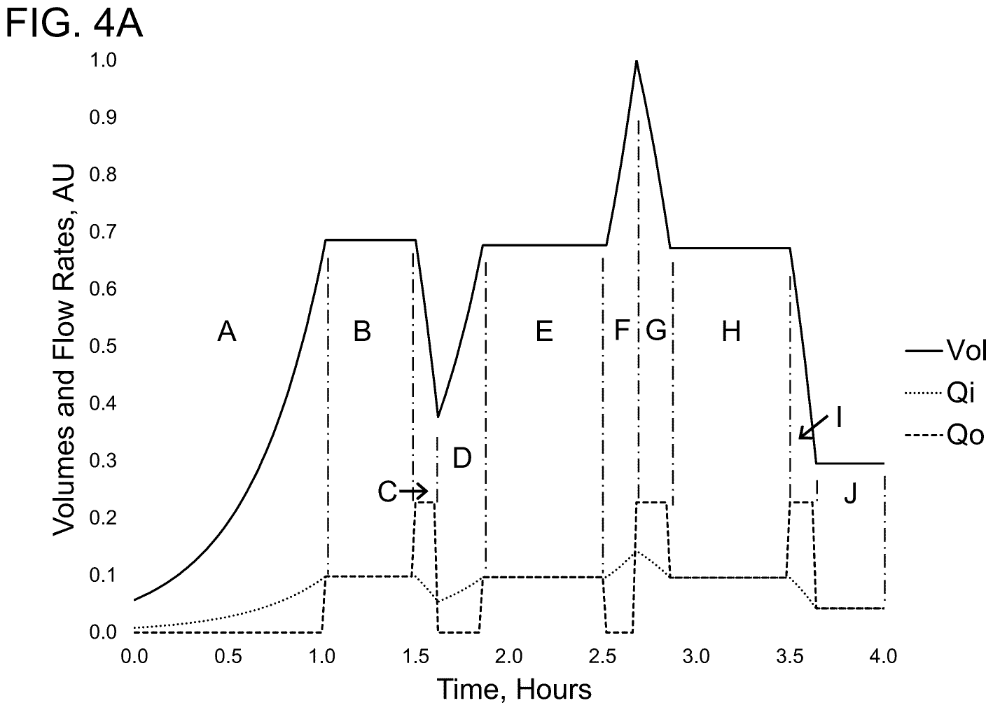

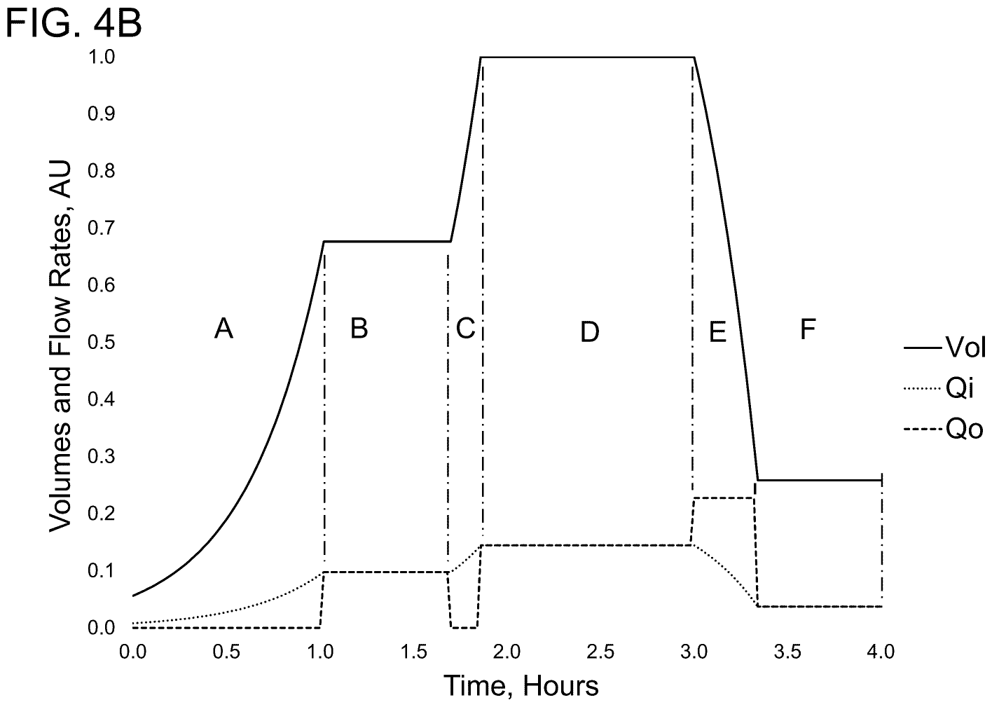

FIGS. 4A-4B show schematically two different time sequences of sample accumulation, storage, and withdrawal in the variable-volume chemostat/bioreactor. FIG. 4A shows how non-perturbative sampling can be accomplished through the time-sequence of the rates of media delivery (Qᵢ), sample accumulation (Vol), and sample withdrawal (Qo) in the variable-volume bioreactor or chemostat/bioreactor according to embodiments of the invention, while FIG. 4B shows sample accumulation followed by a user-specified period of storage of the accumulated sample within the bioreactor. There is a need to accumulate samples for periodic removal, but off-line analysis of samples removed from a standard chemostat could adversely affect the chemical and mechanical microenvironment and violate the chemostasis condition. In contrast, this invention changes the input pump rate, the stirring/mixing rate, and the gas exchange rate so that when the output pump is OFF to accumulate the sample, the delivery pump flow rate, stirring/mixing, and gas exchange are increased so that the media delivery rate is always in proportion to the volume of media and cells in the chemostat/bioreactor. Appropriate adjustments by the master controller of pumping, mixing, and gas exchange rates enable long-term storage of a sample within an SVL bioreactor while maintaining the original chemical and mechanical microenvironmental conditions encountered by cells while in a bioreactor operating at its baseline volume. In FIG. 4A, the sample is first withdrawn from a filled bioreactor and subsequently replaced (B-E), or the bioreactor is overfilled before sample withdrawal (E-H). The phases are labeled as follows: A) Filling phase: No withdrawal, volume increases exponentially with time to maintain chemostat conditions. B) Steady-state chemostat phase: The delivery rate equals the withdrawal rate. C) Sample withdrawal phase: The sample is withdrawn at a constant rate, and the delivery rate decreases as the volume of the bioreactor is decreased, thereby also maintaining the chemostat condition. D) Sample accumulation phase: No withdrawal, delivery rate increases in proportion to the volume to maintain chemical steady state, with slight exponential curvature evident in volume. E) Steady-state chemostat phase, with delivery rate equal to the withdrawal rate. F) Sample accumulation phase: The input flow rates increase with bioreactor volume, and there is no output. G) Sample withdrawal phase: Accelerated sample withdrawal, media delivery remains in proportion to volume to maintain chemostasis, with an almost undetectable curvature. H) Steady-state chemostat phase. The gas mixture and stirring/mixing rate would be regulated as necessary during all phases. The instantaneous volume of media in the bioreactor is shown by the solid line. The dotted and dashed lines show the time course of the delivery and removal of fluid from the bioreactor, respectively. Note that throughout this sequence of events, the input flow rate (solid line), stirring/mixing speed (not shown), and gas exchange rates (not shown) are always proportional or otherwise determined by the instantaneous volume, the defining feature of this invention that ensures that the cellular microenvironment is unperturbed by the accumulation, storage, and removal of metabolically active samples of bioreactor cells and media. The timing and volume of media removed can be specified by the user, and the rate of delivery of replacement media, the stirring/mixing rate, and gas exchange rate are all determined by the master controller so as to ensure a temporally constant cellular microenvironment.

FIG. 4B is similar to the second sampling sequence in FIG. 4A, except that the sample is stored for a user-specified period of time (D) with a delayed sample withdrawal phase (E) that removes an even larger volume than in FIG. 4A. The input and output flow rates are equal and high, consistent with the high volume of media within the bioreactor.

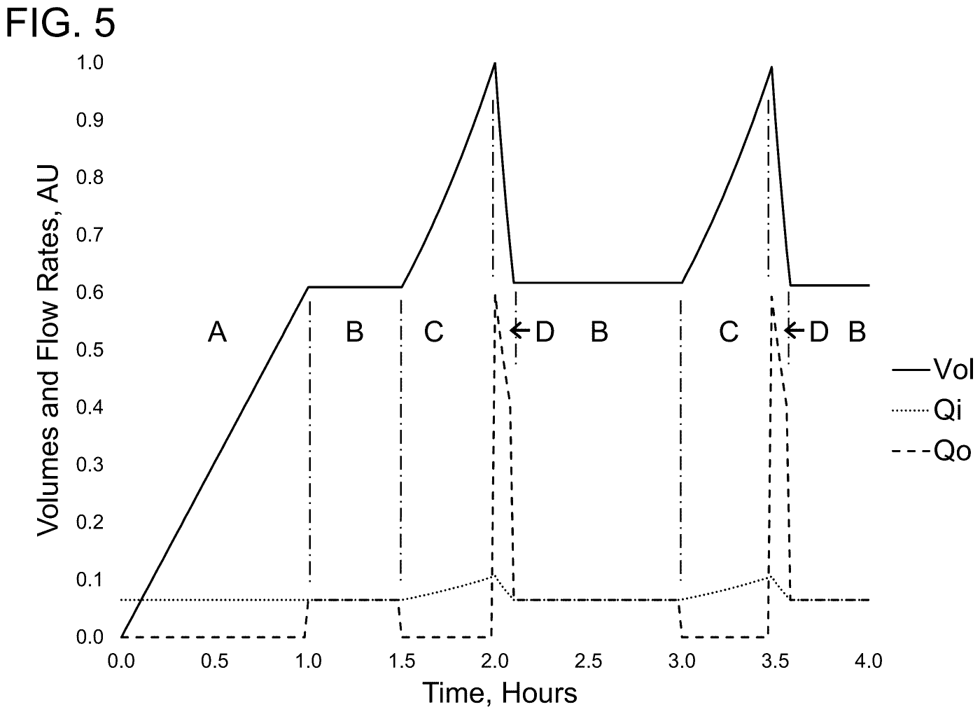

FIG. 5 shows a sequence of events similar to that of FIG. 4A, except that the initial volume increase is linear with time, thereby not satisfying the microenvironmental chemostat condition, but with two master-controller-regulated accumulation and withdrawal phases that do satisfy that condition. This sampling process could be repeated indefinitely. The number of sample aliquots and the volume of each is limited by the starting volume of the bioreactor at the end of phase A and the input media delivery rate. Were media not replaced in this manner, sequential sampling of an SVL bioreactor could quickly empty the bioreactor.

It should be noted that the transition between phases in FIGS. 4A-4B and FIG. 5 would be smooth, but any discontinuous derivatives evident in the graph reflect the discretization of the equations used to generate the traces, not the underlying hydrodynamics.

It is possible, but not shown in FIGS. 4A-4B or FIG. 5, that during the sample collection phase the output flow rate is not zero but instead kept very small to ensure that there are no cells trapped in the output tube during what could be an extended sample accumulation phase, since these cells would still be alive but increasingly deprived of appropriate access nutrients and media. Valves of a particular design could support the washing of cells out of the fluidic lines as necessary.

As shown in the examples presented in FIGS. 4A-4B or FIG. 5, after filling, the chemostat/bioreactor is operated at a constant, initial media delivery rate, Qᵢ, that is determined by the bioreactor volume and the desired dilution rate, and the media removal flow rate, Qₒ, is matched to the input delivery rate.

At time Tₛ, the removal pump is turned off, and the instantaneous delivery rate is determined by the instantaneous volume, V, to produce the same system dilution rate. The rates of gas exchange and stirring/mixing are increased accordingly.

There are a number of different practical considerations in operating a chemostat/bioreactor in a variable-volume, non-perturbative acquisition, storage, or sampling mode. In a classic chemostat, the dilution rate, D, is the ratio of the constant input flow rate Qⁱⁿ divided by the constant volume of the chemostat, V, such that

$$D = {Q}^{in} / V ,$$

where D has units of inverse time, typically inverse hours. The reciprocal of D is the time required to fill an empty chemostat with the flow rate Qⁱⁿ.

In the variable-volume mode, the maximum volume of a sample that can be accumulated and then removed is determined by the difference between the maximum and minimum allowable volumes of the bioreactor,

$${V}_{sample} = {V}_{max} - {V}_{min} ,$$

and the time required to accumulate the sample Tₛₐₘₚₗₑ is simply

$${T}_{sample =} {V}_{sample} / {Q}^{in} .$$

Under the variable-volume paradigm, the instantaneous inflow rate, Qⁱⁿ(t), is proportional to the instantaneous volume of the bioreactor V(t), where the constant of proportionality is simply the dilution rate, D, which is assumed to be constant so as to maintain the same ratio of nutrient delivery per cell independent of the total volume of the cells, such that

$${Q}^{in} ( t ) = D \times V ( t ) .$$

This means that the input flow rate per unit volume is a constant

$${Q}^{in} ( t ) / V ( t ) = D .$$

In a constant volume chemostat or other bioreactor, the input and output flow rates are equal, i.e.,

$${Q}^{in} = {Q}^{out} ,$$

so that Qⁱⁿ−Q^{out}=0, and the volume remains constant.

In the variable-volume chemostat/bioreactor, the input and output flow rates can be different functions of time, such that their difference equals the rate of change of volume of the chemostat/bioreactor, i.e.,

$${Q}^{in} ( t ) - {Q}^{out} ( t ) = dV ( t ) / dt .$$

Hence, if the input flow rate is greater than the output flow rate, then the volume of fluid within the chemostat/bioreactor increases with time. If the output flow rate is greater than the input flow rate, the volume of fluid decreases in time, as illustrated in FIGS. 4A-4B.

Referring to FIG. 2B, which shows one embodiment of the variable-volume chemostat/bioreactor, we see that in steady-state operation, the second output pump is set at a pumping rate greater than the input pump rate, i.e., output overpumping, to ensure that the bioreactor operates at a constant volume. In the chemostat/bioreactor, the maximum bioreactor volume is determined by the height of the overflow withdrawal tube. The shorter media supply tube on the left is designed not to come in contact with the media to avoid back contamination. Starting with an empty chemostat/bioreactor, the fluid level rises at a rate determined by the input pump rate. Once the media rises to make contact with the overflow withdrawal tube, the output pump withdraws media when running. The solution is withdrawn until the meniscus falls below the tube on the right and breaks, allowing the output pump to pump air into the output tube rather than water. The process then repeats, with small fluctuations in chemostat/bioreactor level as small amounts of fluid are added and withdrawn. The extraction volume of each of these small cycles is dependent upon surface tension and contact angle of the tubes, both of which could be adjusted to affect the volume of the extracted bolus of media. Either continuous or pulsatile stirring/mixing can also affect the frequency and volume of the fluid withdrawals, as could the cross-sectional shape of the chemostat/bioreactor reservoir. The spacing of bubbles in the output line will reflect the frequency with which the meniscus is made and broken.

As we will see later, instead of the making and breaking of the meniscus on a withdrawal tube, a system can be configured with a bioreactor level sensor that the master controller could use to determine the pump timing.

In a practical embodiment of a variable-volume chemostat/bioreactor, as might be implemented with the design shown in FIGS. 1A-1C, the periodic collection of a sample involves turning off the output pump periodically so that the fluid level rises inside the chemostat/bioreactor. The input pumping rate is increased in proportion to the instantaneous volume of the chemostat/bioreactor until the desired volume has been accumulated, at which time the output pump is turned on at a significantly higher rate than the input pump (shown in FIG. 2C) to quickly deliver the periodically accumulated samples to the output tube and downstream sensors or collection devices (FIG. 5). As this withdrawal is occurring, the input flow rate has to be decreased accordingly, as shown in FIG. 5. The net result is that samples of cells and media are accumulated within the chemostat/bioreactor, where they maintain the same conditions as the rest of the fluid and cells in the chemostat/bioreactor. Their rapid removal at the end of a possibly lengthy accumulation interval ensures that the biochemistry can be quickly and uniformly stopped during or immediately after the sample removal process.

In order to better appreciate the difference between a fixed-volume chemostat and a variable-volume chemostat/bioreactor, it is useful to revisit the difference between intensive and extensive variables. As an analogous example, the heat capacity of an object is an extensive variable whose value depends upon the mass of the object. The specific heat capacity, or specific heat, is the heat capacity divided by the mass and hence is an intrinsic variable determined by the thermal properties of the material and not the amount of material present. The dilution rate, D, is an intrinsic variable, while a constant input flow rate, Qⁱⁿ, is an extrinsic variable that will need to be large for a large bioreactor and small for a small one. In effect, we have created an intrinsic system variable Qⁱⁿ(t)/V(t), which could be termed a “specific flow rate.” As long as oxygenation and stirring/mixing are scaled similarly, the creation of specific or intrinsic variables is what allows the chemostat/bioreactor to have a variable volume yet a constant chemical and mechanical microenvironment. However, it is important to recognize that the density of the chemostat/bioreactor effluent that contains cells, reduced nutrients, and added metabolites may be higher than the density of the input media, which is free of cells and metabolites. In this context, it may be necessary to account for the difference between mass flow rates and volumetric flow rates. In a classic chemostat, the input mass flow rate must be the same as the output mass flow rate, since mass is conserved in the chemistry, and otherwise the mass of the chemostat would be changing with time. However, if the input density of the input medium is less than the density of the output medium, the volume flow rate at the input will be higher than the volume flow rate at the output. These effects are small, but it may be worthwhile to consider them in the operation of a variable-volume chemostat/bioreactor.

In a classic chemostat, a peristaltic pump delivers input media at a known volumetric flow rate, and a siphon that is part of the fixed volume bioreactor maintains the constant volume within the chemostat. The volumetric output flow rate need not be measured because it is set by the input flow rate and the constant volume; were there a change in density between inflow and outflow, it might not be noticed and with this regulatory method would be irrelevant. Were volumetric pumps used on both the input and the output, flow rates would have to be adjusted to ensure that the volume remained constant.

The variable-volume chemostat/bioreactor has a volume that can change in time, wherein the input and output flow rates are adjusted such that the biomass density and the density flow rates in the variable-volume chemostat/bioreactor are identical to those in the classical chemostat. The biomass density and the density flow rates are intrinsic variables, in that any changes in the volume V (1) of the bioreactor are balanced by the corresponding changes in the flow rates Q (t). Hence, if the classic and variable-volume chemostat/bioreactors have the same intrinsic variables, they have to be biochemically indistinguishable. The variable-volume chemostat/bioreactor has the advantage of providing a means to accumulate a sample without allowing any changes in metabolic state during accumulation.

The same principles can be applied to create a variable-volume turbidistat, wherein the specific optical density of the cells and medium are held constant independent of the volume of fluid contained in the system.

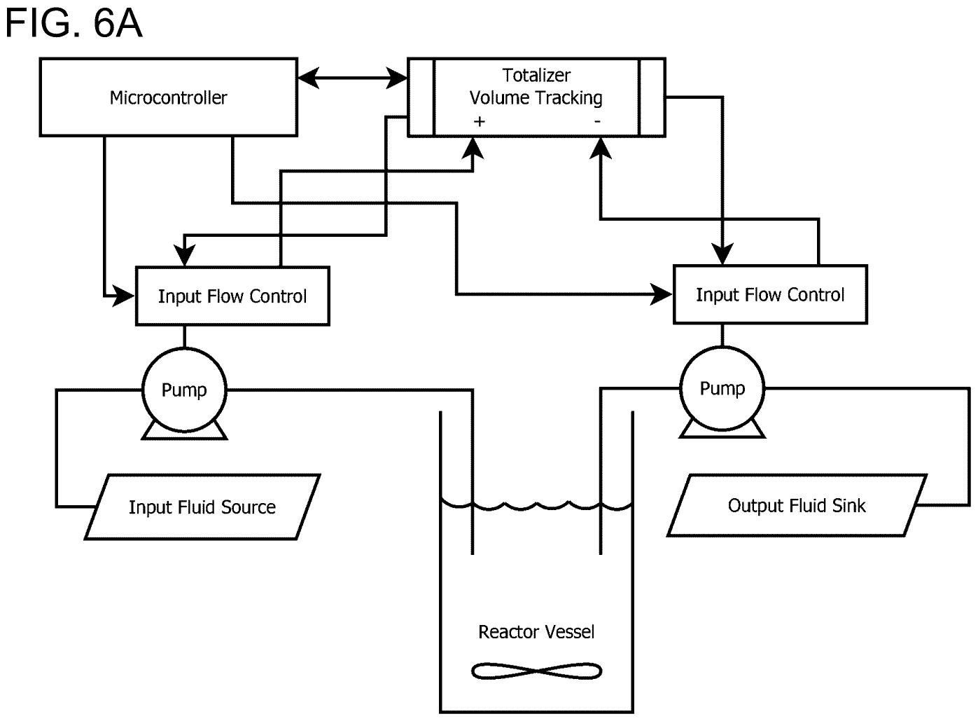

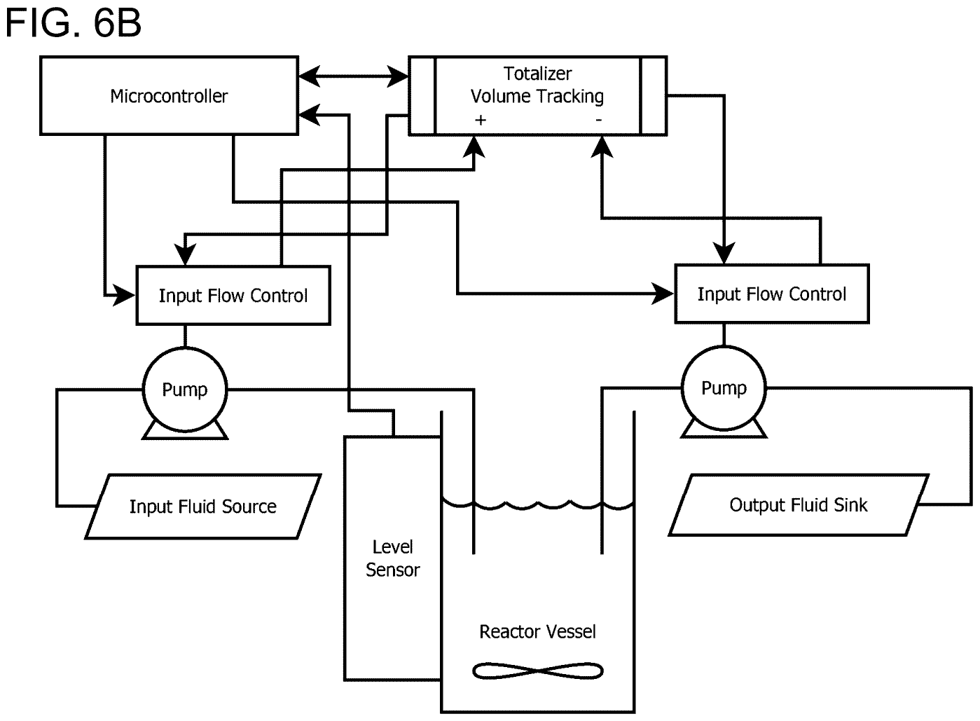

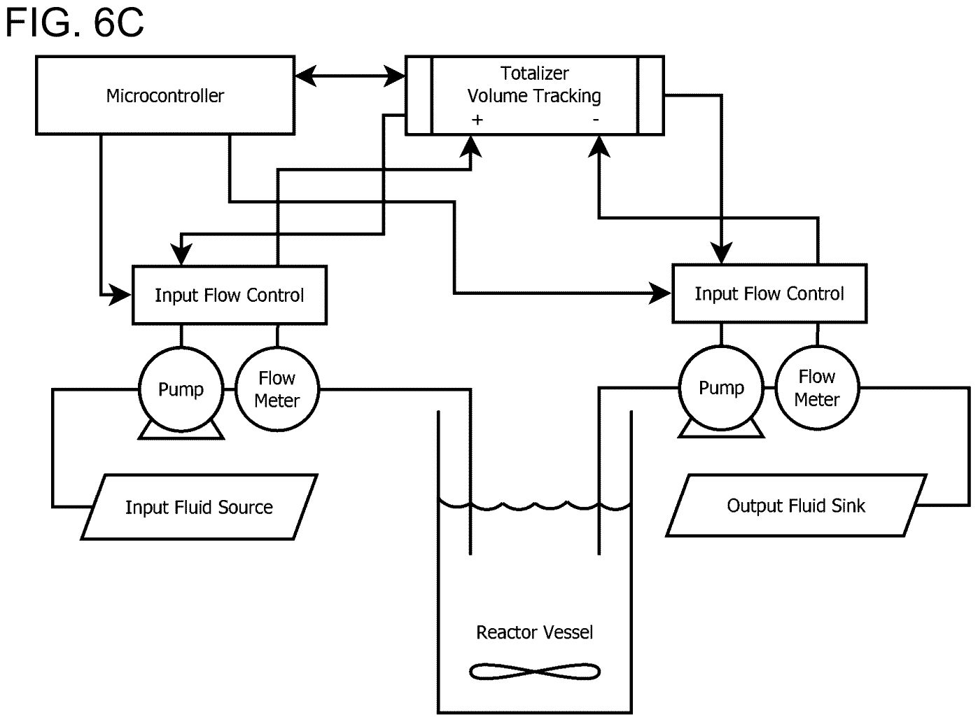

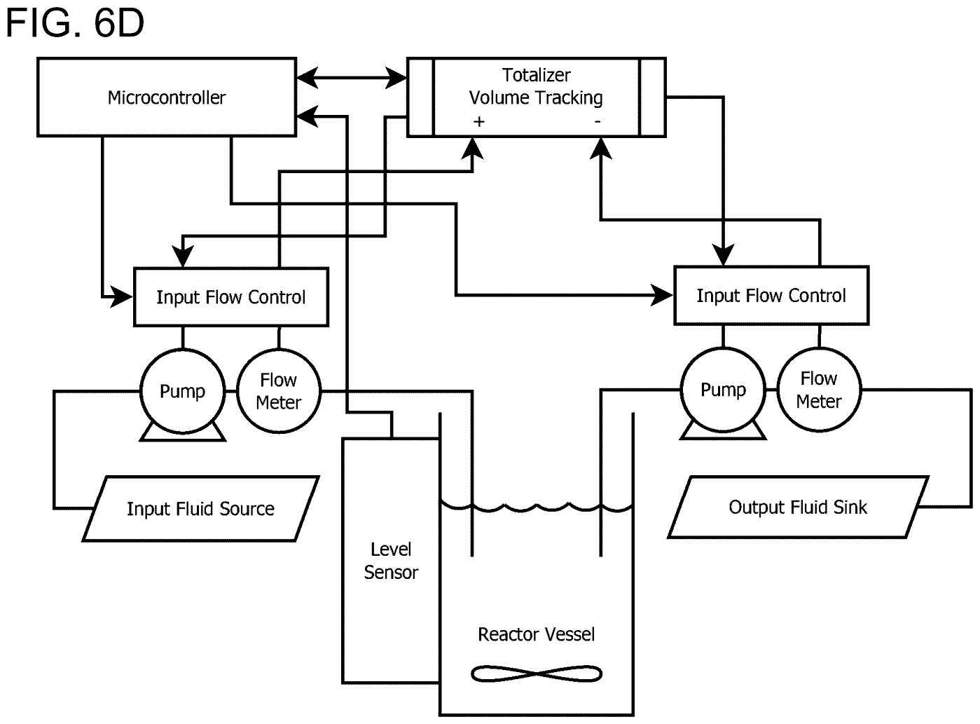

FIGS. 6A-6D show four controller schemes, two with positive displacement pumps (FIG. 6A and FIG. 6B) that can increment or decrement the volume-tracking totalizer within the controller, and two that use centrifugal pumps (FIG. 6C and FIG. 6D), which do not have a direct relation between volume moved and motor rotations but have integral flow sensors that inform the totalizer as to how much fluid was moved into or out of the bioreactor. Two systems do not have bioreactor level sensors (FIG. 6A and FIG. 6C), while two do (FIG. 6B and FIG. 6D). In all cases, gas delivery and removal and stirring/mixing are controlled by the microcontroller. Central to these embodiments is a microcontroller that receives data from the volume-tracking totalizer module and controls the speed of the input and output pumps.

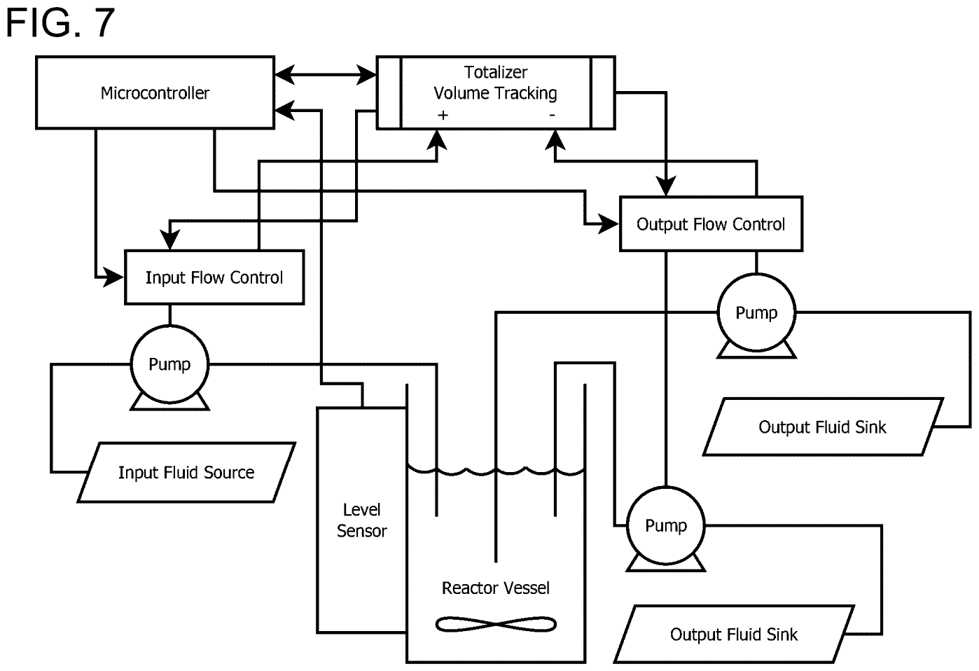

FIG. 7 shows a system similar to that in FIGS. 6A and 6C, with a third pump and sample/split-flow controller used to transfer sample or bulk media with cells to a second reactor vessel to initiate another series of cultures. In this approach, the chemical microenvironment of the parent reactor vessel can be preserved as the sample is transferred to a second reactor vessel for subsequent independent operation and control.

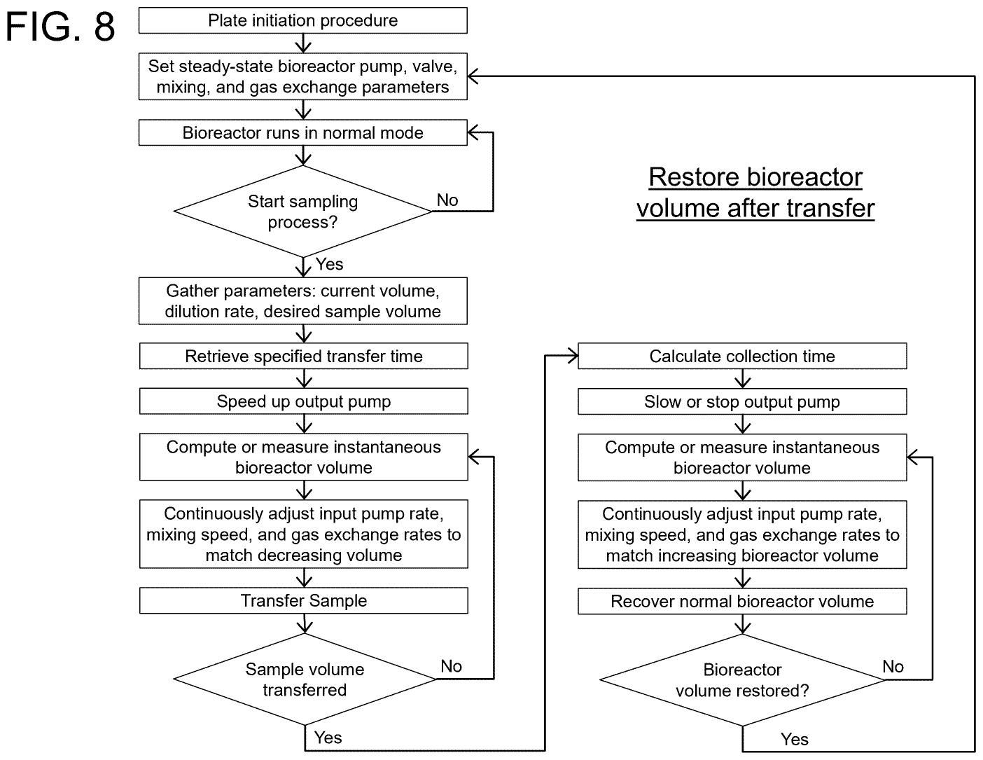

FIG. 8 is a flow chart for the control sequence to be implemented by the master controller system in FIGS. 3A-3B, 6A-6D, and 7 to restore the bioreactor volume after transfer of a sample to another reservoir for off-line analysis or seeding another culture as shown in FIG. 4A intervals C and D. Throughout this process, the master controller uses flow sensors and/or level sensors and commands delivered to positive-displacement pumps to track continuously the instantaneous volume of media and cells within the bioreactor. The instantaneous volume is used to continuously adjust input pump rate, mixing speed, and gas exchange rates accordingly. The steps in FIG. 8 can be visualized using the systems shown in FIG. 3B. The plate initiation procedure involves using the media formulator to selectively withdraw aliquots of media components and deliver them to the media reservoir so that the input pump can deliver media toc the variable-volume bioreactor at a rate proportional to the instantaneous volume of the bioreactor vessel. The master controller in FIG. 3B uses target values and the flow and sensor inputs to control the gas mixture formulator, the media formulator, the stirrer controller and the two output pumps to “set steady-state bioreactor pump, valve, mixing, and gas exchange parameters” in FIG. 8, at which point the bioreactor runs in normal mode. When the sampling process in FIG. 8 is initiated, the subsequent steps are implemented to read the flow, level, gas and other sensors whose output is used to control the rates of the input and output pumps in FIG. 3B. Once the sample has been transferred in FIG. 8, output pump 1 in FIG. 3B is slowed or stopped, and the rate of the input pump is increased in proportion to the instantaneous volume of fluid in the bioreactor. This process evolves until the bioreactor volume is restored and the pump rates are returned to their baseline values.

In each instance, the continuous adjustment can utilize a proportional, integral, and differential (PID) algorithm or other control approaches (not shown) to make these adjustments in a manner that minimizes overshoot, undershoot, and oscillations in the controlled pump speeds. Model-driven control (not shown) with metabolic or gene regulatory models of cellular metabolism could be used to account for the time course of the cellular responses to changes in nutrients, metabolites, growth factors, temperature, pH, and concentrations of dissolved gases. The distinctive features of this invention are the use of a controller and sensors and the decision and control processes in FIG. 8 to continuously maintain the cellular chemical and mechanical microenvironment while samples are being accumulated, stored, and withdrawn from an SVL bioreactor.

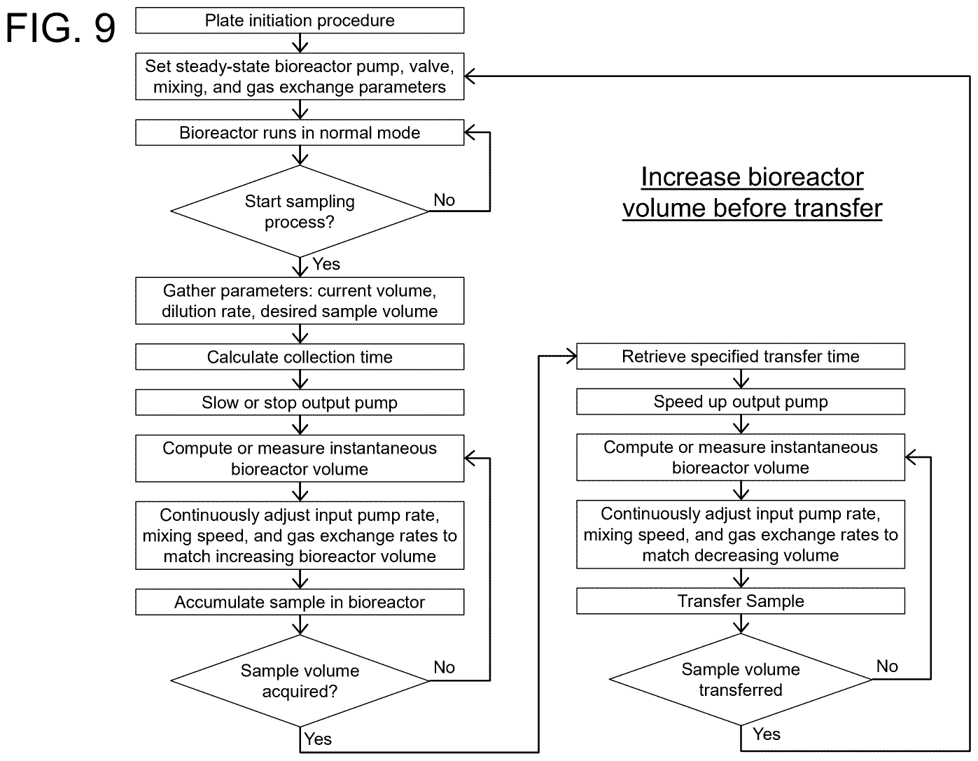

Similar to FIG. 8, FIG. 9 is a flow chart for the control sequence to be implemented by the system in FIGS. 3A-3B, 6A-6D, and 7 to accumulate a sample in a bioreactor prior to its transfer to another reservoir for off-line analysis or seeding another culture as shown in FIG. 4A intervals F and G.

The control steps in FIG. 9 can be illustrated using the input and output pump rates shown in FIG. 4B, where (A) through (F) refer to the phases in FIG. 4B and the words are from the control flow chart:
(A) Plate initiation procedure; set steady-state bioreactor pump, valve, mixing, and gas exchange parameters.
(B) Bioreactor runs in normal mode.
(C) Gather parameters, calculate collection time; slow or stop output pump; compute or measure instantaneous bioreactor volume; continuously adjust input pump rate, mixing speed, and gas exchange rates to match decreasing volume; accumulate sample in bioreactor.
(D) Sample stored for a specified time interval.
(E) Retrieve specified transfer time; speed up output pump; compute or measure instantaneous bioreactor volume; continuously adjust input pump rate, mixing speed, and gas exchange rates to match increasing bioreactor volume.
(F) Recover normal bioreactor volume; set steady-state bioreactor pump, valve, mixing, and gas exchange parameters.
   Each of these steps involve sensors, pumps, and controls in FIG. 3B.

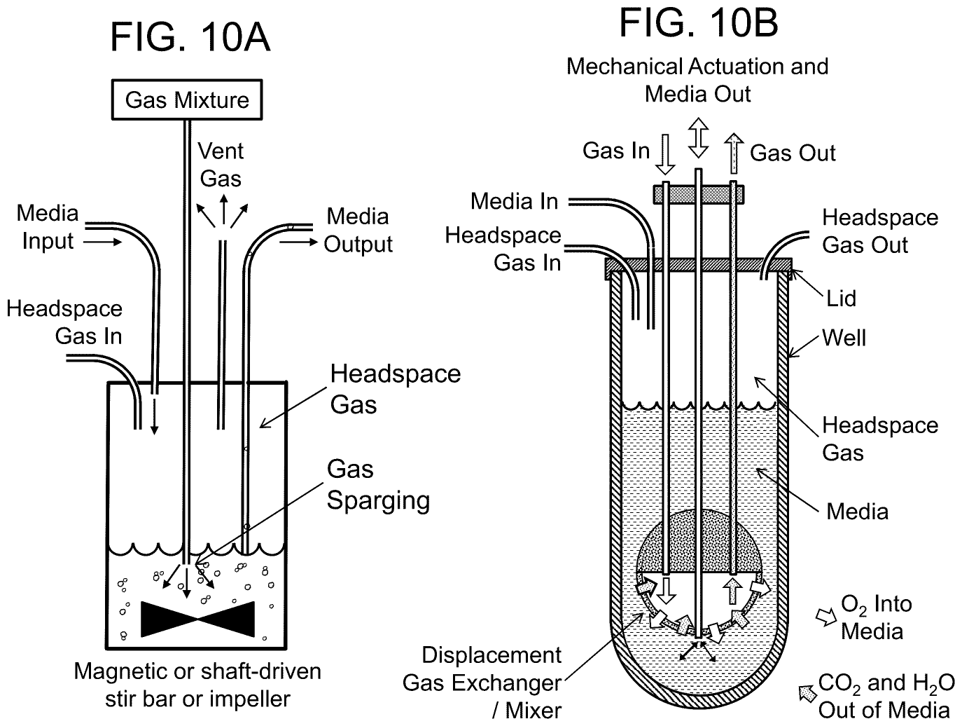

There are a variety of means to stir or mix a bioreactor and control the mixture of gases in both the bioreactor headspace and the stored media. FIGS. 10A-10B show as illustrative examples two methods for mixing and gas exchange in SVL bioreactors. FIG. 10A uses a magnetic or shaft-driven stir bar for mixing, and headspace gas exchange and gas sparging in the liquid for controlling the levels of dissolved gas in the media. FIG. 10B uses a membrane displacement gas exchanger/mixer and headspace gas exchange to both mix and control media gas levels. The gas analyzer and gas mixture formulator in FIG. 3B can be used to measure and control the balance of O₂, CO₂, and N₂, and possibly another inert gas, so as to regulate both the pH and certain cellular biochemical processes in the bioreactor throughout the control process shown in FIGS. 8 and 9. A variety of other methods can be used to control both mixing and gas exchange consistent with continuous maintenance of a constant chemical and mechanical microenvironment during the accumulation, storage, and withdrawal of samples from SVL bioreactors.

The processes covered by this invention can run for extended periods of time, including days to weeks to months, as long as there is an adequate supply of nutrients and control is maintained as described. In such cases, the system can then be operated as a continuous bioreactor whose effluent delivers cells and media containing the target product molecule. Intermittent accumulation, storage, and withdrawal of additional cells and media can be superimposed on this continuous process as part of the bioreactor control scheme.

We also note that the control schemes described by this invention could be applied to abiotic and other chemical reactors where different chemicals are added and mixed and samples removed over time, but without the presence of living cells.

Without intent to limit the scope of the invention, further details of a classical constant volume chemostat/bioreactor and novel variable-volume chemostat/bioreactors according to the embodiments of the invention are given below. Note that titles or subtitles may be used in the examples for convenience of a reader, which in no way should limit the scope of the invention. Moreover, certain theories are proposed and disclosed herein; however, in no way should they, whether they are right or wrong, limit the scope of the invention so long as the invention is practiced according to the invention without regard for any particular theory or scheme of action.

### The Classical Constant Volume Chemostat

A chemostat bioreactor is a continuously stirred tank reactor (CSTR) for which the volumetric inflow rate of input media, Qⁱⁿ, is the same as the volumetric outflow rate of the bioreactor's media, Q^{out}, and the volume of the bioreactor, V, is constant over time. Due to chemostats being well-stirred, the outflow media concentrations and biomass concentration are assumed to be the same as the reactor media concentrations, cⱼ where j=1, 2, . . . , and the reactor biomass concentration, c_{biomass}. The inflow media concentrations,

$${c}_{j}^{in}$$

where j=1, 2, . . . , typically only contains essential nutrients and one or more substrates, such as glucose. Biomass

$${c}_{b i o m a s s}^{i n} = 0.$$

The differential equation for a CSTR is given as

$$\frac{d [ {c}_{j} V ]}{dt} = {Q}^{i n} {c}_{j}^{i n} - {Q}^{out} {c}_{j} + {R}_{j} V ,$$

where Rⱼ is the net reaction rate. In the case of constant volume, this equation reduces to

$$\frac{d {c}_{j}}{dt} = \frac{{Q}^{i n}}{V} {c}_{j}^{i n} - \frac{{Q}^{o u t}}{V} {c}_{j} + {R}_{j} .$$

At a steady state, the biochemistry within the chemostat does not change with time. Substrate-limited chemostats can control the stable steady states of the reactor's media concentrations by changing the inflow concentration of substrates. In this case, the physical density of cells in the media or the optical density of the cell-containing media will reflect the extent to which limiting the supply of a particular substrate in the media affects the cell's metabolic activity, allowing the determination of how growth rate is affected by the concentration of a particular substrate whose concentration is reduced below “baseline” levels.

The production rate of biomass is referred to as the growth rate, μ. The growth rate is heavily dependent on substrate concentration. The steady-state growth rate, μₛₛ, in a substrate-limited chemostat is equal to the volumetric outflow rate divided by the reactor volume. This ratio,

$$\frac{{Q}^{o u t}}{V} ,$$

is referred to as the dilution rate. If the dilution rate is greater than the maximum ratio, growth rate, then washout occurs where the biomass approaches zero. The differential equation describing biomass concentration over time is

$$\frac{d {c}_{b i o m a s s}}{dt} = ( μ - \frac{{Q}_{o u t}}{V} ) {c}_{b i o m a s s} .$$

To summarize, a chemostat is a continuous stirred tank reactor (CSTR) such that

$${Q}^{i n} = {Q}^{out} and \frac{d V}{dt} = 0 .$$

At the steady-state,

$$\frac{d {c}_{j}}{dt} = 0 , ∀ j ,$$

$$\frac{d {c}_{b i o m a s s}}{dt} = 0 ,$$

$${μ}_{s s} = \frac{{Q}^{o u t}}{V} if \frac{{Q}^{o u t}}{V} \leq max μ , and$$

$${μ}_{s s} = 0 if \frac{{Q}^{o u t}}{V} \geq max μ .$$

Variable-Volume Chemostat/Bioreactor with Approximated Volume Differential Equation

Chemostats can have heterogeneous processes, such as Saccharomyces cerevisiae producing gaseous rather than dissolved carbon dioxide from aqueous glucose during ethanol fermentation. However, typically chemostats are treated as liquid-phase systems with water as the solvent. For liquid-phase reactors with excess solvent, the differential equation for reactor volume is approximated as

$$\frac{d V}{dt} = {Q}^{i n} - {Q}^{out} .$$

This equation can be used to derive a more generalized treatment of the substrate-limited chemostat to allow for variable volume and let Qⁱⁿ not necessarily be equal to Q^{out}. Using previous equations, it can be shown that

$$\frac{d [ V {c}_{b i o m a s s} ]}{d t} = ( μ V - {Q}^{out} ) {c}_{b i o m a s s}$$

$$\to {c}_{b i o m a s s} \frac{d V}{d t} + V \frac{d {c}_{b i o m a s s}}{d t} = ( μ V - {Q}^{out} ) {c}_{b i o m a s s}$$

$$\to {c}_{b i o m a s s} ( {Q}^{i n} - {Q}^{out} ) + V \frac{d {c}_{b i o m a s s}}{d t} = ( μ V - {Q}^{out} ) {c}_{b i o m a s s}$$

$$\to V \frac{d {c}_{b i o m a s s}}{d t} = ( μ V - {Q}^{i n} V ) {c}_{biomass}$$

$$\to \frac{d {c}_{b i o m a s s}}{d t} = ( μ - \frac{{Q}^{out}}{V} ) {c}_{b i o m a s s} .$$

This result differs from the original differential equation for c_{biomass} since the former depends on Q^{out} while the latter depends on Qⁱⁿ. The term “dilution rate” is more difficult to use in this case, since

$$\frac{{Q}^{i n}}{V}$$

is not necessarily equal to

$$\frac{{Q}^{o u t}}{V} .$$

For this reason, the rest of this discussion will not use the term “dilution rate.” The steady-state growth rate for the variable-volume case is given as

$${μ}_{s s} = \frac{{Q}^{i n}}{V} if \frac{{Q}^{i n}}{V} \leq max μ , and {μ}_{s s} = 0 if \frac{{Q}^{i n}}{V} \geq max μ .$$

With this new model, we can consider a case where the bioreactor is at steady-state, and we want to increase the volume while keeping

$$\frac{d {c}_{b i o m a s s}}{dt} = 0 .$$

This can be achieved as long as

$${Q}^{i n} = μ V .$$

Variable-Volume Chemostat/Bioreactor with Constant Mass Densities

Qⁱⁿ=μV only holds if we assume the volume differential equation has the form of

$$\frac{d V}{dt} = {Q}^{i n} - {Q}^{out} .$$

The more generalized form of the volume differential equation is given as

$$\frac{d [ ρ V ]}{dt} = {Q}^{in} {ρ}^{in} - {Q}^{out} ρ ,$$

where ρ=c_{biomass}M_{biomass}+ΣⱼcⱼMⱼ is the mass density of all the components of the reactor with M_{biomass} and Mⱼ being molecular weights, and

$${ρ}^{in} = {c}_{biomass}^{in} {M}_{biomass} + {\sum }_{j} {c}_{j}^{in} {M}_{j}$$

is the mass density of all the components of the input media. In the case that the total mass density is constant, ρ=ρⁱⁿ, this equation reduces to

$$\frac{dV}{dt} = {Q}^{in} - {Q}^{out} .$$

Variable-Volume Chemostat/Bioreactor with Non-Constant Mass Densities

For non-constant densities, ρ≠ρⁱⁿ, it is useful to utilize an equation of state, f(c_{biomass}, c₁, c₂, . . . )=0, to derive a new differential equation for volume. Equations of state relate the thermodynamic properties (temperature, pressure, volume, composition, etc.) to each other.

$$1 = {c}_{biomass} {\overline{V}}_{biomass} + {\sum }_{j} {c}_{j} {\overline{V}}_{j} ,$$

where V̅_{biomass} and V̅ⱼ are partial molar volumes.

The equation of state can be used to express the volume differential equation as

$$\frac{dV}{dt} = \frac{{Q}^{in}}{ϕ} ( {f}_{biomass} {c}_{biomass}^{in} + {\sum }_{j} {f}_{j} {c}_{j}^{in} ) - {Q}^{out} + \frac{V}{ϕ} ( {f}_{biomass} μ + {\sum }_{j} {f}_{j} {R}_{j} ) ,$$

$$where ϕ = {f}_{biomass} {c}_{biomass} + {\sum }_{j} {f}_{j} {c}_{j} , {f}_{j} = \frac{\partial f}{\partial {c}_{j}} , and {f}_{biomass} = \frac{\partial f}{\partial {c}_{biomass}} .$$

To simplify notation, let

$$α = \frac{1}{ϕ} ( {f}_{biomass} {c}_{biomass}^{in} + {\sum }_{j} {f}_{j} {c}_{j}^{in} ) and β = \frac{1}{ϕ} ( {f}_{biomass} μ + {\sum }_{j} {f}_{j} {R}_{j} ) .$$

With this new notation,

$$\frac{dV}{dt} = α {Q}^{in} - {Q}^{out} + β V .$$

α describes how input media and bioreactor media affect the equation of state. If there is no difference in these effects, then α=1. In the last term, β describes how the chemistry in the bioreactor affects the equation of state. If chemistry has no effect on the equation of state, then β=0.

If we assume c_{biomass} is constant, then,

$$\frac{dV}{dt} = μ V - {Q}^{out} .$$

Consequently, the volumetric inflow rate required to increase the bioreactor volume while keeping biomass concentration constant with time is given as

$${Q}^{in} = \frac{μ - β}{α} V .$$

### Utilizing the Specific Volume Equation of State

The partial molar volumes are functions of temperature, pressure, and composition. If ideal mixing is assumed, then the partial molar volumes become specific volumes which are functions of only temperature and pressure. With the ideal mixing assumption, the equation of state becomes

$$1 = {c}_{biomass} {V}_{biomass}^{°} + {\sum }_{j} {c}_{j} {V}_{j}^{°} , where {V}_{biomass}^{°}$$

is the specific volume of pure biomass and

$${V}_{j}^{°}$$

is the specific volume of pure component j.

We assume that the equation of state is

$$f = {c}_{biomass} {V}_{biomass}^{°} + {\sum }_{j} {c}_{j} {V}_{j}^{°} - 1 , with {f}_{j} = {V}_{j}^{°} , and {f}_{biomass} = {V}_{biomass}^{°} . Consequently , α = {\sum }_{j} {V}_{j}^{°} {c}_{j}^{in} and β = {V}_{biomass}^{°} μ + {\sum }_{j} {V}_{j}^{°} {R}_{j} .$$

The volumetric inflow rate to maintain constant biomass is given as

$${Q}^{in} = \frac{μ}{φ} ( V - {V}_{biomass} ) - \frac{V}{φ} {\sum }_{j} {V}_{j}^{°} {R}_{j} where φ = {\sum }_{j} {V}_{j}^{°} {c}_{j}^{in} .$$

### Utilizing the Partial Molar Volume Equation of State

$${Q}^{in} = \frac{μ}{φ} ( V - {V}_{biomass} ) - \frac{V}{φ} {\sum }_{j} {V}_{j}^{°} {R}_{j}$$

can be generalized further by using partial molar volumes instead of specific volumes for the equation of state. Using partial molar volumes for the equation of state gives

$$f = {c}_{biomass} {\overline{V}}_{biomass} + {\sum }_{j} {c}_{j} {\overline{V}}_{j} - 1 with {f}_{j} = {V}_{j} \frac{\partial {\overline{V}}_{j}}{\partial {c}_{j}} + {c}_{biomass} \frac{\partial {\overline{V}}_{biomass}}{\partial {c}_{biomass}} + {\sum }_{i \neq j} {C}_{i} \frac{\partial {V}_{i}}{\partial {c}_{j}} and {f}_{biomass} = {V}_{biomass} \frac{\partial {\overline{V}}_{biomass}}{\partial {c}_{biomass}} + {\sum }_{j} {c}_{j} \frac{\partial {\overline{V}}_{j}}{\partial {c}_{biomass}} .$$

From this, α, β, and Qⁱⁿ can be calculated and simplified if it is known that the partial molar volumes for a given media component are independent of a set of media components.

From this more detailed analysis, we conclude once again that the concept of a variable-volume chemostat/bioreactor is viable. Modern computer-controlled pumps can definitely adjust their pumping rate with sufficient accuracy and speed to be able to track and account for the increase in media volume within the chemostat/bioreactor as a future sample is accumulated and stored within the chemostat/bioreactor. This approach eliminates the sources of error and uncertainty that arise from attempting to collect samples whose volumes represent a significant fraction of the chemostat/bioreactor volume and whose biochemistry can be difficult to halt as cells are collected over long periods of time. This will become particularly important as instruments are built that contain a large number of very small, sample-volume-limited chemostat/bioreactors.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

What is claimed is:

1. A non-perturbative sampling system, comprising:
a bioreactor comprising a bioreactor chamber containing media with cells for cell maintenance, growth, and division; an input tube coupled to the chamber for delivering nutrient-laden media that supports the cell maintenance, growth, and division within the chamber; and an output tube coupled to the chamber for withdrawing a sample from the chamber;
an input pump coupled to the input tube and configured to operably deliver, by the input tube, the nutrient-laden media at an inflow rate;
an output pump coupled to the output tube and configured to operably withdraw, by the output tube, the sample at an outflow rate; and
a controller configured to operate the input pump and the output pump to regulate the inflow rate and the outflow rate, respectively, such that the bioreactor is a variable-volume bioreactor in which an instantaneous volume of media either varies continuously with time or is held constant, and operable in a sample accumulation phase, a sample withdrawal phase, or a volume restoration phase, wherein the sample withdrawal phase either follows the sample accumulation phase or precedes the volume restoration phase, wherein the inflow rate increases in proportion to an increase of the instantaneous volume of media in the chamber in the sample accumulation phase and decreases in proportion to a decrease of the instantaneous volume of the chamber in the sample withdrawal phase, so as to maintain the quantity of nutrients per cell within the media in the chamber unchanged.

2. The system of claim 1, wherein the input pump is a positive displacement pump, or a non-metering pump with an in-line flow sensor including a flow sensor coupled to the input tube and/or a level sensor coupled to the chamber, for controlling the delivery of a predetermined volume of media to the chamber.

3. The system of claim 1, wherein the output pump is a positive displacement pump, or a non-metering pump with an in-line flow sensor including a flow sensor coupled to the output tube, for controlling the withdrawal of a predetermined volume of media and cells from the chamber.

4. The system of claim 1, further comprising an additional output pump coupled to an overflow withdrawal tube that is coupled to the chamber and configured to operably set a maximum volume of media in the chamber.

5. The system of claim 1, wherein the controller is further configured to maintain a dilution rate that is a ratio of the inflow rate divided by the instantaneous volume being a constant, so as to maintain a same ratio of nutrient delivery per cell independent of the total volume of the media and the cells that it contains.

6. The system of claim 1, wherein a maximum volume of the sample that is accumulated and then removed is determined by a difference between a maximum allowable volume and a minimum allowable volume of the chamber, and the time required to accumulate the sample is determined by the maximum volume of the sample divided by the inflow rate.

7. The system of claim 1, wherein the inflow rates and the outflow rate are adjustable simultaneously to support different phases of sample accumulation, sample withdrawal, and volume restoration.

8. The system of claim 1, wherein the inflow rate and the outflow rates are of different functions of time such that a difference between the inflow rate and the outflow rate equals a rate of change of the instantaneous volume with time.

9. The system of claim 8, wherein when the inflow rate is greater than the outflow rate, then the volume of media within the chamber increases with time, and when the outflow rate is greater than the inflow rate, the volume of fluid decreases in time.

10. The system of claim 1, wherein the bioreactor is operable in a filling phase during which the outflow rate is zero and the inflow rate is greater than zero so that the instantaneous volume increases with time, or in a steady-state phase during which the outflow rate is same as the inflow rate.

11. The system of claim 1, wherein the output pump is turned off or otherwise substantially reduced at the beginning of the sample accumulation phase and turned on or otherwise substantially increased at the beginning of the sample withdrawal phase.

12. The system of claim 1, wherein the bioreactor further comprises a means for stirring/mixing and oxygenating the media in the chamber respectively at a stirring/mixing rate and a gas exchange rate to ensure that the media within the chamber is well mixed, uniformly oxygenated, and at a desired pH over a full range of volumes of media contained in the chamber during all phases of operation.

13. The system of claim 12, wherein the stirring/mixing rate, the gas exchange rate, and an input gas mixture composition are adjustable so as to ensure that local conditions throughout the chamber remain unchanged over the full range of volumes of media contained in the bioreactor chamber during all phases of operation.

14. The system of claim 12, wherein growth conditions, and nutrient and gas concentrations within the entire media in the chamber are maintained at original conditions by modulating the inflow rate, the outflow rate, the gas exchange rate and the stirring/mixing rate in a manner that maintains static biochemical conditions independent of the instantaneous volume of media and cells within the chamber.

15. The system of claim 12, wherein a fraction of the media in the chamber is withdrawable without prior accumulation, but with the inflow rate, the outflow rate, the gas exchange rate and the stirring/mixing rate modulated post-withdrawal in a manner that maintains static biochemical conditions independent of the instantaneous volume of cells and media within the chamber.

16. The system of claim 12, wherein by the dynamic control of the inflow rate, the outflow rate, the gas exchange rate, and the stirring/mixing rate, any arbitrary volume within the chamber can be maintained at the same biochemical state as any volume within an industry-standard, constant-volume bioreactor is achievable.

17. A method for operating a bioreactor with a variable volume, wherein the variable-volume bioreactor is characterized with a media volume that varies with time in a chamber, an inflow rate at which nutrient-laden media is delivered into the bioreactor, such that the media composition stays constant over time, and an outflow rate at which a sample is withdrawn from the bioreactor, comprising:
regulating the inflow rate, the outflow rate, and the input gas mixture such that the bioreactor operates in a sample accumulation phase, a sample withdrawal phase, or a volume restoration phase, wherein the sample withdrawal phase either follows the sample accumulation phase or precedes the volume restoration phase, wherein for the sample withdrawal phase, the outflow rate higher than the inflow rate to withdraw the sample volume from the chamber rapidly at one time and the inflow rate and gas exchange rate remain in proportion to the instantaneous volume to maintain chemostasis in the chamber; wherein for the volume restoration phase, the outflow rate is zero or very small and the inflow rate and gas exchange rate increase in proportion to the instantaneous volume of media in the chamber so as to restore the volume without changes in a metabolic state within the chamber.

18. The method of claim 17, wherein a dilution rate that is a ratio of the inflow rate divided by the instantaneous volume is a constant, so as to maintain the same ratio of nutrient delivery per cell independent of the total volume of the media and cells.

19. The method of claim 17, further comprising stirring/mixing and oxygenating the media in the chamber respectfully at a stirring/mixing rate and a gas exchange rate to ensure that the media within the chamber is well mixed, uniformly oxygenated, and has the desired carbon dioxide levels.

20. The method of claim 19, wherein the gas exchange rate and the stirring/mixing rate are adjustable so as to ensure that the local conditions throughout the bioreactor chamber remain unchanged.
